(19) United States
(12) Patent Application Publication
(10) Pub. No.: US 2006/0078921 A1
(43) Pub. Date: Apr. 13, 2006

(54) Biomarkers and expression profiles for toxicology

(76) Inventors: Franziska Boess, Riehen (CH); Laura Suter-Dick, Bottmingen (CH); Detlef Wolf, Grenzach-Wyhlen (DE)

Correspondence Address: HOFFMANN-LA ROCHE INC.;PATENT LAW DEPARTMENT, 340 KINGSLAND STREET, NUTLEY, NJ, 07110, US

(21) Appl. No.: 11/224,663

(22) Filed: Sep. 12, 2005

Related U.S. Application Data
(62) Division of application No. 10/388,934, filed on Mar. 14, 2003, now abandoned.

(30) Foreign Application Priority Data
Mar. 14, 2002 (EP) 02005336.9
Jul. 17, 2002 (EP) 02015657.6

(51) Int. Cl. C12Q 1/68

(52) U.S. Cl. 435/6

(57) ABSTRACT

The present invention is based on the determination of the global changes in gene expression in tissues or cells exposed to known toxins, in particular hepatotoxins, as compared to unexposed tissues or cells as well as the identification of individual genes that are differentially expressed upon toxin exposure. The invention includes methods of predicting at least one toxic effect of a compound, predicting the progression of a toxic effect of a compound, and predicting the hepatoxicity of a compound. Also provided are methods of predicting the mechanism of toxicity of a compound. In a further aspect, the invention provides probes comprising sequences that specifically hybridize to genes in Table 3 as well as solid supports comprising at least two of the said probes.

### PRIORITY TO RELATED APPLICATIONS

This application is a division, of U.S. application Ser. No. 10/388,934, filed Mar. 14, 2003, now pending, which claims the benefit of European Application No. 02005336.9, filed Mar. 14, 2002 and European Application No. 02015657.6 filed Jul. 17, 2002. The entire contents of the above-identified applications are hereby incorporated by reference.

### BACKGROUND OF THE INVENTION

The present invention relates to toxicogenomic methods useful in the development of safe drugs. More specifically, the present invention relates to methods for the prediction of a toxic effect, especially hepatotoxicity, in animal models or cell cultures. Furthermore, expression profiles characteristic of different mechanisms of hepatoxicity as well as specific markers for hepatoxicity are provided.

The gene expression pattern governs cellular development and physiology, and is affected by pathological situations, including disease and the response to a toxic insult. Bearing this in mind, it becomes clear that the study of gene and protein expression in preclinical safety experiments will help toxicologists to better understand the effects of chemical exposure on mammalian physiology. On the one hand, the identification of a certain number of modulated genes and/or proteins after exposure to a toxicant will lead to the identification of novel predictive and more sensitive biomarkers which might replace the ones currently used. The knowledge regarding marker genes for particular mechanisms of toxicity, together with the rapidly growing understanding of the structure of the human genome will form the basis for the identification of new biomarkers. These markers may allow the prediction of toxic liabilities, the differentiation of species-specific responses and the identification of responder and non-responder populations. Gene expression analysis is an extremely powerful tool for the detection of new, specific and sensitive markers for given mechanisms of toxicity (Fielden, M. R., and Zacharewski, T. R. (2001). Challenges and limitations of gene expression profiling in mechanistic and predictive toxicology. Toxicol Sci 60, 6-10). These markers should provide additional endpoints for inclusion into early animal studies, thus minimising the time, the cost and the number of animals needed to identify the toxic potential of a compound in development. Also, this will lead to the development of relevant screening assays in vivo and/or in vitro. The understanding of the molecular mechanisms underlying toxicity will also provide more insight into species-specific response to drugs and should immensely increase the predictability of potential risk accumulation for drug-combinations or drug-disease interactions. Moreover, chemically induced changes in gene expression are likely to occur at exposures to chemicals below those that induce an adverse toxicological outcome. As drug-induced liver toxicity is a major issue for health care and drug development, great interest lies in hepatotoxins. Currently, the predictivity of gene and protein expression for toxicity is a generally accepted assumption supported by some published results, but substantially more data are needed to prove the validity of this hypothesis (Waring, J. F., Ciurlionis, R., Jolly, R. A., Heindel, M., and Ulrich, R. G. (2001). Microarray analysis of hepatotoxins in vitro reveals a correlation between gene expression profiles and mechanisms of toxicity. Toxicol Lett 120, 359-68; Bulera, S. J., Eddy, S. M., Ferguson, E., Jatkoe, T. A., Reindel, J. F., Bleavins, M. R., and De La Iglesia, F. A. (2001). RNA expression in the early characterization of hepatotoxicants in Wistar rats by high-density DNA microarrays. Hepatology 33, 1239-58; Bartosiewicz M. J., Jenkins, D., Penn, S., Emery, J., and Buckpitt, A. (2001). Unique gene expression patterns in liver and kidney associated with exposure to chemical toxicants. J Pharmacol Exp Ther 297, 895-905). A widespread approach to validate these new tools is the use of model compounds in animal models to produce expression profiles which are expected to be characteristic for the compound under examination. Model compounds that have been used for gene expression profiling in the liver include WY-14,643, phentobarbital, clofibrate, ethanol and acetaminophen. The majority of the published results confirm the regulation of genes previously identified and add a large number of genes modulated by the test compound. Thus microarrays showed the induction of cytochromes (CYP2B and CYP3A) as well as of genes related to apoptosis and DNA-repair by phentobarbital (Carver, M. P., and Clancy, B. (2000). Transcriptional profiling of phenobarbital (PB) hepatotoxicity in the mouse. Toxicological Sciences 54, 383). Similarly, studies on the peroxisome proliferator WY-14,643 showed the induction of CYP4A, GST and acyl-CoA hydroxylase, as well as of genes associated with oxidative damage, with cell proliferation and with apoptosis (Carfagna, M. A., Baker, T. K., Wilding, L. A., Neeb, L. A., Torres, S., Ryan, T. P., and Gelbert, L. M. (2000). Effects of a peroxisome proliferator (WY-14,643) on hepatocyte transcription using microarray technology. Toxicological Sciences 54, 383). Ruepp and co-workers investigated gene expression changes after treating mice with acetaminophen, and found that genes such as metallothioneins, c-fos, glutathione peroxidase and proteasome-related-genes were induced (Ruepp, S., Tonge, R. P., Wallis, N. T., Davison, M. D., Orton, T. C., and Pognan, F. (2000). Genomic and proteomic investigations of acetaminophen (APAP) toxicity in mouse liver in vivo. Toxicological Sciences 54, 384). Similar results were also presented by Suter et al. (Suter, L., Boelsterli, U. A., Winter, M., Crameri, F., Gasser, R., Bedoucha, M., deVera, C., and Albertini, S. (2000). Toxicogenomics: Correlation of acetaminophen-induced hepatotoxicity with gene expression using DNA microarrays. Toxicological Sciences 54, 383) and by Reilly et al. (Reilly, T. P., Bourdi, M., Brady, J. N., Pise-Masison, C. A., Radonovich, M. F., George, J. W., and Pohl, L. R. (2001). Expression profiling of acetaminophen liver toxicity in mice using microarray technology. Biochem Biophys Res Commun 282, 321-8). So far, expression profiles and toxicity markers were only provided for specific model compounds in the prior art. Therefore, the technical problem underlying the present invention was to provide for gene expression profiles and toxicity markers, which are characteristic not only for a specific toxic compound, but for a specific mechanism of toxicity and which are reproducible.

As can be seen, there is a need for methods for the prediction of toxic effects of a compound, for the prediction of the mechanism of toxicity of a compound, especially for the prediction of hepatotoxicity, by using reproducible gene expression profiles caused by known toxic compounds, gene expression profiles characteristic of a mechanism of hepatoxicity, and specific marker genes.

### SUMMARY OF THE INVENTION

The present invention is based on the determination of the global changes in gene expression in tissues or cells exposed to known toxins, in particular hepatotoxins, as compared to unexposed tissues or cells as well as the identification of individual genes that are differentially expressed upon toxin exposure.

The invention includes methods of predicting at least one toxic effect of a compound, predicting the progression of a toxic effect of a compound, and predicting the hepatoxicity of a compound. Also provided are methods of predicting the mechanism of toxicity of a compound. In a further aspect, the invention provides probes comprising sequences that specifically hybridize to genes in Table 3 as well as solid supports comprising at least two of the said probes, and primers for specific amplification of the genes of Table 3. The prediction of toxic effects comprises the steps of a) generating a database with the expression of marker genes elicited by known toxic compounds in animal models or cell culture systems, b) obtaining a biological sample from the model systems; c) obtaining a gene expression profile characteristic of a given toxicity mechanism and/or detecting and/or measuring the expression of (a) specific marker gene(s) d) comparing the expression profile and/or expression of specific marker gene(s) with the database of step a).

More specifically, in one aspect of the present invention, a method of predicting at least one toxic effect of a compound, comprises detecting the level of expression of on or more genes from Table 3 in a tissue or cell sample exposed to the compound; wherein differential expression of the one or more genes from Table 3 is indicative of at least one toxic effect.

In another aspect of the present invention, a method of predicting at least one toxic effect of a compound comprises (a) detecting the level of expression of one or more genes from Table 3 in a tissue or cell sample exposed to the compound; and (b) comparing the level of expression of the one or more genes to their level of expression in a control tissue or cell sample, wherein differential expression of the one or more genes in Table 3 is indicative of at least one toxic effect.

In yet another aspect of the present invention, a method of predicting the progression of a toxic effect of a compound comprises detecting the level of expression in a tissue or cell sample exposed to the compound of one or more genes from Table 3, wherein differential expression of the one or more genes in Table 3 is indicative of toxicity progression.

In a further aspect of the present invention, a method of predicting the mechanism of toxicity of a compound comprises detecting the level of expression in a tissue or cell sample exposed to the compound of one or more genes from Table 3, wherein differential expression of the one or more genes in Table 3 is associated with a specific mechanism of toxicity.

In still a further aspect of the present invention, a method of predicting at least one toxic effect of a compound comprises detecting the level of expression of one of the genes selected from Table 4 in a tissue or cell sample exposed to the compound, wherein differential expression of the gene selected from Table 4 is indicative of at least one toxic effect.

In still a further aspect of the present invention, a set of nucleic acid primers have primers that specifically amplify at least two of the genes from Table 3.

In still a further aspect of the present invention, a set of nucleic acid probes have probes that comprise sequences which hybridize to at least a specific number of the genes from Table 3. While not being limited thereto, the specific number of genes may be at least 2 genes from Table 3, at least 5 genes from Table 3, and at least 10 genes from Table 3.

In still a further aspect of the present invention, a solid support comprises at least two probes, wherein each of the probes comprises a sequence that specifically hybridizes to a gene in Table 3.

In still a further aspect of the present invention, a computer system comprises a database containing DNA sequence information and expression information of at least two of the genes from Table 3 from tissue or cells exposed to a hepatotoxin, and a user interface.

In still a further aspect of the present invention, a computer system for predicting at least one toxic effect of a compound comprises a processor and a memory coupled to the processor; wherein the memory stores a first set of data including the level of expression of one or more genes from Table 3 in a tissue or cell sample exposed to the compound, and the memory stores a second set of data including the level of expression of the one or more genes from Table 3 in a control tissue or cell sample; and the processor compares the first set of data with the second set of data to predict the at least one toxic effect of the compound.

In yet a further aspect of the present invention, a kit comprises 1) at least one solid support having at least two probes, wherein each of the probes comprises a sequence that specifically hybridizes to a gene in Table 3, and 2) gene expression information for the said genes.

These and other features, aspects and advantages of the present invention will become better understood with reference to the following drawings, description and claims.

### BRIEF DESCRIPTION OF THE DRAWINGS

FIG. 1: Effect of CCl4 on PEG-3 (progression elevated gene-3) expression and circulating ALT (alanine aminotransferase) levels. Expression levels are expressed in arbitrary units. Circulating ALT-levels are expressed in μkat/ml. For each dose group (Control, Low dose=0.25 ml/kg and High dose=2 ml/kg) the values obtained for each of the 5 animals are represented.

FIG. 2: Effect of HDZ on PEG-3 expression. The median expression level of the gene PEG-3 for 10 control animals, 10 low-dose animals (10 mg/kg) and 10 high dose animals (60 mg/kg) are represented. Expression levels are expressed in arbitrary units. An expression of below 100 is considered as non-detectable.

FIG. 3: Effect of NFT on PEG-3 expression and on circulating ALT (alanine aminotransferase) levels. The median expression level of the gene PEG-3 for 10 control animals, 10 low-dose animals (5 mg/kg), 10 mid-dose animals (20 mg/kg) and 10 high dose animals (60 mg/kg) are represented. Expression levels are expressed in arbitrary units. An expression of below 100 is considered non-detectable

FIG. 4: Effect of Thioacetamide and Thioacetamide-S-oxide on PEG-3 expression at 3 different time points by in vitro exposure of hepatocytes. Rat primary hepatocytes (in monolayer cultures) were exposed to 0, 30, 100 and 300 μM Thioacetamide-S-Oxide and to 3 and 10 mM Thioacetamide. Expression level of PEG-3 were at 2, 6 and 24 hs after exposure. For each dose and time point triplicate samples were analyzed. The expression level of PEG-3 in each experimental condition is displayed in FIG. 4A through C, where each line represents the expression level for each replicate. Expression levels are expressed in arbitrary units. In addition, cytotoxicity (LDH-release) of Thioacetamide-S-oxide at several doses and time points is represented in FIG. 4D.

FIG. 5: Effect of in vitro exposure of hepatocytes to Thioacetamide (TAA) and Thioacetamide-S-oxide (TSO) on the expression levels of PEG-3 and on some co-regulated genes as determined by duster analysis. Rat primary hepatocytes (in monolayer cultures) were exposed to 0, 30, 100 and 300 μM Thioacetamide-S-Oxide and to 3 and 10 mM Thioacetamide and analyzed at 3 different time points. Each line represents a single gene; the intensity of the grey colour is proportional to the expression level.

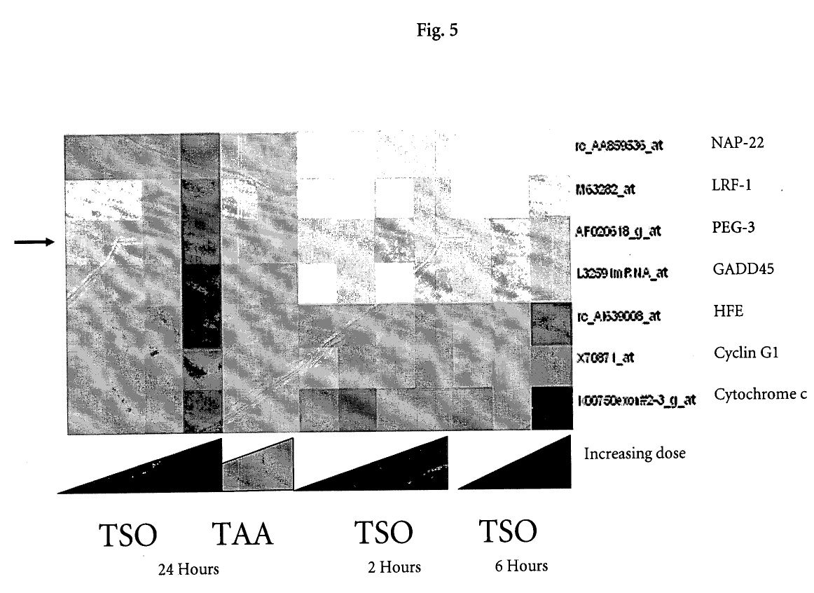

FIG. 6: Effect of in vitro exposure to Glucokinase activators on the expression levels of PEG-3, GADD-45 and GADD-153 and on mitochondrial beta-oxidation. FIG. 6A represents the induction of PEG-3, GADD-45 and GADD-153 with increasing doses of Ro 28-2310 at 6 hours. FIG. 6B shows the inhibition of beta-oxidation by 3 glucokinase activators.

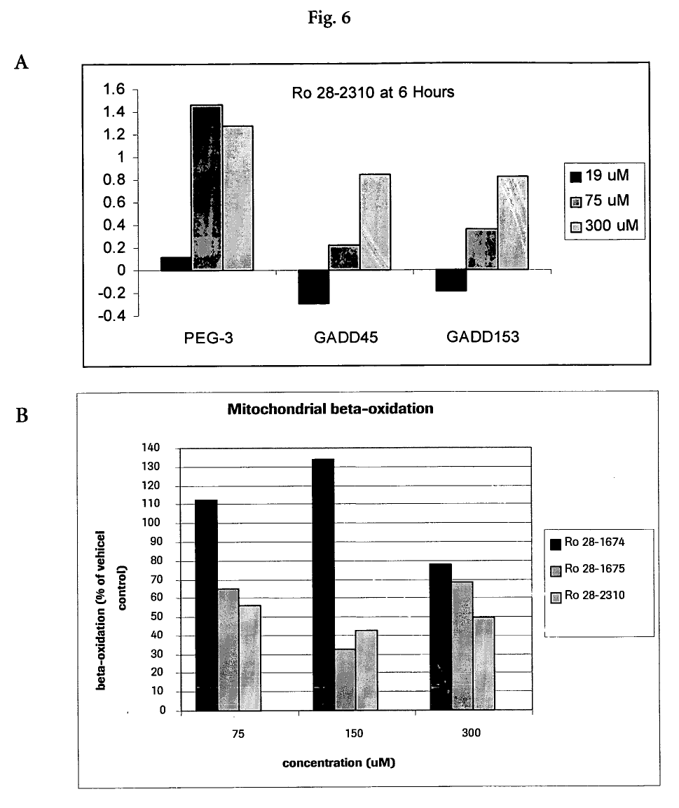

FIG. 7: Recursive Feature Elimination (RFE) for generation of support vector machines (SVMs) for the direct acting group (see Example 9).

FIG. 8: Classification of Amineptine as a steatotic compound. The bigger a positive discriminant value is, the better is the data fit into a specific class defined by the respective SVM. A negative discriminant value means that data do not fit into a compound class.

FIG. 9: Classification of 1,2-Dichlorobenzene as a direct acting compound. The bigger a positive discriminant value is, the better is the data fit into a specific class defined by the respective SVM. A negative discriminant value means that data do not fit into a compound class.

FIG. 10: Differentiation of toxic and non-toxic compounds using RT-PCR

FIG. 11: Western-Blots of liver extracts with antibody specific for CYP2B. Two representative animals from each treatment group were analyzed. a: lane 1: MW-markers; lanes 2 and 3: control (6H); lanes 4 and 5: Ro 65-7199 (6H); lanes 6 and 7: Ro 66-0074 (6H); lanes 8 and 9: controls (24H); lanes 10 and 11: Ro 65-7199 (24H); lanes 12 and 13: Ro 66-0074 (24H). b: lane 1: MW-markers; lanes 2 and 3: controls (7 Days); lanes 4 and 5 30 mg/kg/d Ro 65-7199 (7 Days); lanes 6 and 7: 100 mg/kg/d Ro 65-7199 (7 Days); lanes 8 and 9: 400 mg/kg/d Ro 65-7199 (7 Days); lane 11: Positive control (phenobarbital induced rat microsomal extract). Inlet bargraphs represent the densitometric quantification of each gel.

In the present invention it was found that marker genes are differentially expressed in tissues obtained after exposure of non-human animals, e.g. rats, to model toxic compounds at doses and/or time points in which these compounds did not elicit the conventionally measured response of elevation in plasma liver enzymes. It was also observed that this elevation of particular marker genes was evident not only after in vivo exposure of the test animals but also in vitro in primary hepatic cell cultures exposed to a similar group of hepatotoxins. Furthermore, the regulation of a group of genes by several compounds with a similar mechanism of toxicity provides a characteristic gene expression profile or “fingerprint” for said mechanism of toxicity.

In the present study, compounds with well characterized toxicity as Chlorpromazine, Cyclosporine A, Erythromycin, Glibenclamide, Lithocholic acid, Ro 48-5695 (Endothelin receptor antagonist), Dexamethasone, 1,2-Dichlorobenzene, Aflatoxin B1, Bromobenzene, Carbon tetrachloride, Didofenac, Hydrazine, Nitrofurantoin, Thioacetamide, Concanavaline A, Tacrine, Tempium (Lazabemide), Tolcapone (Tasmar), 1,4-Dichlorobenzene, Amineptin, Amiodarone, Doxycycline, Ro 28-1674 (glucokinase activator), Ro 28-1675 (glucokinase activator), Ro 65-7199 (5-HT6 receptor antagonist), Tetracycline, Dinitrophenol, Cyproterone Acetate, Phenobarbital, Clofibrate, Acetaminophen, Thioacetamide-S-Oxide, Perhexiline, Methapyrilene were selected as known hepatotoxins. These compounds are widely known to cause hepatic injury in animals and/or in man, as described in “Toxicology of the liver, 2^{nd}. Ed, Ed. By G. L. Plaa and W. R. Hewitt, Target organ toxicology series, 1997. A brief summary of the known effects of these compounds is listed below.

Carbon tetrachloride (CCl4), bromobenzene and 1,2-dichlorobenzene are halogenated, highly reactive compounds leading to toxicity in the liver in rodents and in man (Brondeau M T, Bonnet P, Guenier J P, De Ceaurriz J. (1983). Short-term inhalation test for evaluating industrial hepatotoxicants in rats. Toxicol Lett. 19, 139-46; Rikans, L. E. (1989). Influence of aging on chemically induced hepatotoxicity: role of age-related changes in metabolism. Drug Metab Rev 20, 87-110). For CCl4, several studies have shown that the toxicity is mediated by its metabolic product, the highly reactive trichloromethyl free radical (Stoyanovsky, D. A., and Cederbaum, A. I. (1999). Metabolism of carbon tetrachloride to trichloromethyl radical: An ESR and HPLC-EC study. Chem Res Toxicol 12, 730-6). This radical leads to lipid peroxidation and can react with cellular proteins and with DNA (Castro, G. D., Diaz Gomez, M. I., and Castro, J. A. (1997). DNA bases attack by reactive metabolites produced during carbon tetrachloride biotransformation and promotion of liver microsomal lipid peroxidation. Res Commun Mol Pathol Pharmacol 95, 253-8). Secondary liver injury following the administration of these halogenated compounds is believed to be caused by inflammatory processes originating from products of activated Kupffer cells (Edwards, M. J., Keller, B. J., Kauffman, F. C., and Thurman, R. G. (1993). The involvement of Kupffer cells in carbon tetrachloride toxicity. Toxicol Appl Pharmacol 119, 275-9). Thus, the observed toxicity is due to direct action of the free radicals and to indirect action mediated by cytokines such as TNF alpha (DeCicco, L. A., Rikans, L. E., Tutor, C. G., and Hornbrook, K. R. (1998). Typical lesions produced by these compounds a few hours after a single administration are centrilobular cell degeneration and necrosis accompanied by lipid peroxidation, followed by hepatic regeneration starting 48 hours after administration. Elevation of serum enzyme activities is seen as a result of of the hepatocellular necrosis (e.g. AST, ALT, SDH).

Hydrazine and hydrazine derivatives are among the early drugs reported to cause damage to the liver. Thioacetamide and its metabolite Thioacetamide-S-oxide are also known to cause liver injury, histopathological examination showed necrotic hepatocytes around the central vein with infiltration of macrophages, neutrophils and eosinophils. Thus biochemical and histologic and clinical features indicate hepatocellular injury, with parenchimal degeneration and necrosis (Dashti, Jeppsson, Hagerstrand, Hultberg, Srinivas, Abdulla, Joelsson and Bengmark (1987). Early biochemical and histological changes in rats exposed to a single injection of thioacetamide. Pharmacol Toxicol 3, 171-4.; Albano, Goria-Gatti, Clot, Jannone and Tomasi (1993). Possible role of free radical intermediates in hepatotoxicity of hydrazine derivatives. Toxicol Ind Health 3, 529-38.).

Cyclosporine A (CsA) is an immunosupressant that has been reported to induce cholestasis in transplanted patients. Several mechanisms have been proposed to explain this toxic manifestation: hepatotoxicity, competition for bilirary excretion, inhibition of bilirubin excretion, inhibition of the synthesis of bile acids, etc. (Le Thai, Dumont, Michel, Erlinger and Houssin (1988). Cholestatic effect of cyclosporine in the rat. An inhibition of bile acid secretion. Transplantation 4, 510-2.). In spite of the liver being one of the main target organs for CsA-induced toxicity, the kidney is also a target organ for toxicity. Nevertheless, nephrotoxicity seems to be the consequence of chronic exposure to the drug. Using animal studies (rats), it has been shown that the bile flow is significantly reduced after chronic (3 weeks) or acute (single dose) administration of CsA. This decrease in BA-flow is reflected by an increase in plasma bile acids and plasma bilirubin. No histopathological findings accompany this effect (Stone, Warty, Dindzans and Van Thiel (1988). The mechanism of cyclosporine-induced cholestasis in the rat. Transplant Proc 3 Suppl 3, 841-4, Roman, Monte, Esteller and Jimenez (1989). Cholestasis in the rat by means of intravenous administration of cyclosporine vehicle, Cremophor EL. Transplantation 4, 554-8).

Tacrine is a compound for the treatment of Alzheimer's disease in man. In treated patients, it shows hepatotoxicity with an incidence of 40-50%. In rodents, tacrine elicited hepatic toxicity manifested as pericentral necrosis and fatty changes, accompanied by an increase in circulating liver enzymes (Monteith and Theiss (1996). Comparison of tacrine-induced cytotoxicity in primary cultures of rat, mouse, monkey, dog, rabbit, and human hepatocytes. Drug Chem Toxicol 1-2, 59-70; Stachlewitz, Arteel, Raleigh, Connor, Mason and Thurman (1997). Development and characterization of a new model of tacrine-induced hepatotoxicity: role of the sympathetic nervous system and hypoxia-reoxygenation. J Pharmacol Exp Ther 3, 1591-9).

Concanavaline A is a model compound used for studying the role of liver-associated T cells in acute hepatitis produced in rats. Concanavalin A produces a severe hepatitis, which can be assessed by serum biochemistry showing increased interleukines (IL-6 and TNF-alpha), as well as alanine aminotransferase (ALT) (Mizuhara, O'Neill, Seki, Ogawa, Kusunoki, Otsuka, Satoh, Niwa, Senoh and Fujiwara (1994). T cell activation-associated hepatic injury: mediation by tumor necrosis factors and protection by interleukin 6. J Exp Med 5, 1529-37).

Chlorpromazine has a clear and well-studied profile of producing liver injury in man. It is the most extensively studied neuroleptic and the hepytic injury that produces is hepatocanalicular cholestasis. Up to 1% of the treated patients develop jaundice. Some studies have shown that chlorpromazine inhibits Na+-K+-ATPase cation pumping in intact cells, therefore contributing to the chlorpromazine-induced cholestasis in animals and humans. (Van Dyke and Scharschmidt (1987). Effects of chlorpromazine on Na+-K+-ATPase pumping and solute transport in rat hepatocytes. Am J Physiol 5 Pt 1, G613-21.). Lithocholic acid is one of the bile acids transported into the bile canaliculi. An increase in the concentration of lithocholic acid causes intrahepatic cholestasis (Shefer, Zaki and Salen (1983). Early morphologic and enzymatic changes in livers of rats treated with chenodeoxycholic and ursodeoxycholic acids. Hepatology 2, 201-8).

Erythromycine have been incriminated as the cause of cholestatic liver injury. The pattern of injury is usually hepatocanalicular cholestasis. In rare casis, erythromycin can also lead to liver necrosis. (Gaeta, Utili, Adinolfi, Abernathy and Giusti (1985). Characterization of the effects of erythromycin estolate and erythromycin base on the excretory function of the isolated rat liver. Toxicol Appl Pharmacol 2, 185-92.). Glibenclamide has been associated with reversible cholestasis in clinical case studies (Del-Val, Garrigues, Ponce and Benages (1991). Glibenclamide-induced cholestasis. J Hepatol 3, 375).

Dinitrophenol is a widely used model compound for mitochondrial uncoupling. Dosing of animals with this compound leads to increased mitochondrial respiration, decreased ATP-levels and increase in body temperature (Okuda, Lee, Kumar and Chance (1992). Comparison of the effect of a mitochondrial uncoupler, 2,4-dinitrophenol and adrenaline on oxygen radical production in the isolated perfused rat liver. Acta Physiol Scand 2, 159-68).

Dexamethasone is a known glucocorticoid which is used in many experimental models to induce the activity of cytochromes P450 in the liver and in hepatocyte cultures (Kocarek and Reddy (1998). Negative regulation by dexamethasone of fluvastatin-inducible CYP2B expression in primary cultures of rat hepatocytes: role of CYP3A. Biochem Pharmacol 9, 1435-43). Other drugs that are usually related with hepatomegaly and/or peroxisome proliferation and are known inducers of some cytochromes P450 in the liver and in hepatocyte cell cultures are phenobarbital, cyproterone acetate and fibrates such as clofibrate (Menegazzi, Carcereri-De Prati, Suzuki, Shinozuka, Pibiri, Piga, Columbano and Ledda-Columbano (1997). Liver cell proliferation induced by nafenopin and cyproterone acetate is not associated with increases in activation of transcription factors NF-kappaB and AP-1 or with expression of tumor necrosis factor alpha. Hepatology 3, 585-92.; Kietzmann, Hirsch-Ernst, Kahl and Jungermann (1999). Mimicry in primary rat hepatocyte cultures of the in vivo perivenous induction by phenobarbital of cytochrome P-450 2B1 mRNA: role of epidermal growth factor and perivenous oxygen tension. Mol Pharmacol 1, 46-53; Diez-Fernandez, Sanz, Alvarez, Wolf and Cascales (1998). The effect of non-genotoxic carcinogens, phenobarbital and clofibrate, on the relationship between reactive oxygen species, antioxidant enzyme expression and apoptosis. Carcinogenesis 10, 1715-22).

Acetaminophen is a widely used analgesic and antipyretic drug that causes acute liver damage upon overdosis. This drug is often missused for suicidal purposses. If overdosed, the hepatic glutathione pool becomes depleted and the metabolic activation of the compound leads to a highly reactive metabolite. This metabolite can bind to DNA and proteins in the cell, leading to hepatocellular necrosis (Tarloff, Khairallah, Cohen and Goldstein (1996). Sex- and age-dependent acetaminophen hepato- and nephrotoxicity in Sprague-Dawley rats: role of tissue accumulation, nonprotein sulfhydryl depletion, and covalent binding. Fundam Appl Toxicol 1, 13-22; Cohen and Khairallah (1997). Selective protein arylation and acetaminophen-induced hepatotoxicity. Drug Metab Rev 1-2, 59-77; Fountoulakis, Berndt, Boelsterli, Crameri, Winter, Albertini and Suter (2000). Two-dimensional database of mouse liver proteins: changes in hepatic protein levels following treatment with acetaminophen or its nontoxic regioisomer 3-acetamidophenol. Electrophoresis 11, 2148-61).

Methapyrilene is an antihistamin drug that causes acute periportal hepatotoxicity in rats, but also exerts a variety of toxic effects in the liver. Apparently, CYP2C11 is responsible for the suicide substrate bioactivation of methapyrilene and the acute toxicologic outcome largely relied upon an abundance of detoxifying enzymes present in the liver Another potentially very significant effect of MP is that it induces a large increase in hepatic cell proliferation coupled with mitochondrial proliferation. In addition, some results suggest that methapyrilene hydrochloride is a DNA damaging agent (Althaus, Lawrence, Sattler and Pitot (1982). DNA damage induced by the antihistaminic drug methapyrilene hydrochloride. Mutat Res 3-6, 213-8; Ratra, Cottrell and Powell (1998). Effects of induction and inhibition of cytochromes P450 on the hepatotoxicity of methapyrilene. Toxicol Sci 1, 185-96).

Tetracyclines and Doxycyclines lead to dose-dependent hepatic injury. The hepatotoxicity of tetracyclines is well known. The characteristic lesion is microvesicular steatosis which poor prognosis, resembling Reye's syndrome. The underlying mechanism of toxicity seems to be the inhibition of mitochondrial beta oxidation together with an inhibition of the transport of lipids from the liver (Hopf, Bocker and Estler (1985). Comparative effects of tetracycline and doxycycline on liver function of young adult and old mice. Arch Int Pharmacodyn Ther 1, 157-68; Lienart, Morissens, Jacobs and Ducobu (1992). Doxycycline and hepatotoxicity. Acta Clin Belg 3, 205-8).

Diclofenac is a widely used NSAID (non-steroid anti-inflammatory drug). Several cases related hepatic injury, sometimes with fatal outcome, with the administration of this compound. The The pattern of injury is usually hepatocellular with acute necrosis. The mechanism by which diclofenac elicits this effect is unknown, but some speculations have been made regarding metabolic idiosyncracy. Also, diclofenac can bind irreversibly to hepatic proteins via its acyl glucuronide metabolite; these protein adducts could be involved in the pathogenesis of diclofenac-associated liver damage (Kretz-Rommel and Boelsterli (1994). Mechanism of covalent adduct formation of diclofenac to rat hepatic microsomal proteins. Retention of the glucuronic acid moiety in the adduct. Drug Metab Dispos 6, 956-61).

Nitrofurantoin is an antimicrobial widely used in the treatment of urinary tract infection which is known to cause acute and chronic liver injury. The injury can be either cholestatic or hepatocellular, and the underlying mechanism seems to be immunologic idiosyncrasy (Villa, Carugo and Guaitani (1992), No evidence of intracellular oxidative stress during ischemia-reperfusion damage in rat liver in vivo. Toxicol Lett 2-3, 283-90; Tacchini, Fusar-Poli and Bernelli-Zazzera (2002). Activation of transcription factors by drugs inducing oxidative stress in rat liver. Biochem Pharmacol 2, 139-148).

Aflatoxin B1 is a contaminant in food, source: Aspergillus flavus and Aspergillus parasiticus. Aflatoxin induces also ROS production, lipid peroxidation and 8-OhdG formation in DNA. It reacts also with various liver and blood plasma proteins, particularly with serum albumin. Acutelly, it leads to liver necrosis, given chronically shows a carcinogenic effect (Liu, Yang, Lee, Shen, Ang and Ong (1999). Effect of Salvia miltiorrhiza on aflatoxin B1-induced oxidative stress in cultured rat hepatocytes. Free Radic Res 6, 559-68; Barton, Hill, Yee, Barton, Ganey and Roth (2000). Bacterial lipopolysaccharide exposure augments aflatoxin B(1)-induced liver injury. Toxicol Sci 2, 444-52).

Amineptine, amiodarone and perhexiline are drugs known to cause microvesicular steatosis through the inhibition of mitochondrial beta oxidation (Le Dinh, Freneaux, Labbe, Letteron, Degott, Geneve, Berson, Larrey and Pessayre (1988). Amineptine, a tricyclic antidepressant, inhibits the mitochondrial oxidation of fatty acids and produces microvesicular steatosis of the liver in mice. J Pharmacol Exp Ther 2, 745-50; Bach, Schultz, Cohen, Squire, Gordon, Thung and Schaffner (1989). Amiodarone hepatotoxicity: progression from steatosis to cirrhosis. Mt Sinai J Med 4, 293-6; Deschamps, DeBeco, Fisch, Fromenty, Guillouzo and Pessayre (1994). Inhibition by perhexiline of oxidative phosphorylation and the beta-oxidation of fatty acids: possible role in pseudoalcoholic liver lesions. Hepatology 4, 948-61; Fromenty and Pessayre (1997). Impaired mitochondrial function in microvesicular steatosis. Effects of drugs, ethanol, hormones and cytokines. J Hepatol Suppl 2, 43-53).

In the present invention it was found that the modulation of gene expression by several compounds that show a similar hepatotoxicity defines a characteristic profile which is expected to be similar for further compounds that elicit the same type of toxicity. Thus, these profiles can be used for the prediction of the toxic potential of unknown compounds. Said characteristic profiles (or “fingerprints) for classes of hepatotoxins are defined in Table 3.

Accordingly, the present invention relates to a method of predicting at least one toxic effect of a compound, comprising detecting the level of expression of one or more genes from Table 3 in a tissue or cell sample exposed to the compound, wherein differential expression of the genes in Table 3 is indicative of at least one toxic effect.

The present invention moreover provides a method of predicting at least one toxic effect of a compound, comprising:
(a) detecting the level of expression of one or more genes from Table 3 in a tissue or cell sample exposed to the compound;
(b) comparing the level of expression of the genes to their level of expression in a control tissue or cell sample, wherein differential expression of the genes in Table 3 is indicative of at least one toxic effect.

In a further embodiment, the present invention relates to a method of predicting the progression of a toxic effect of a compound, comprising detecting the level of expression in a tissue or cell sample exposed to the compound of one or more genes from Table 3, wherein differential expression of the genes in Table 3 is indicative of toxicity progression.

As defined in the present invention, a toxic effect includes any adverse effect on the physiological status of a cell or an organism. The effect includes changes at the molecular or cellular level. A preferred toxic effect is hepatotoxicity, which includes pathologies comprising among others liver necrosis, hepatitis, fatty liver and cholestasis.

The progression of a toxic effect is defined as the histological, functional or physiological manifestation with time of a toxic injury that can be detected by measuring the gene expression levels found after initial exposure of an animal or cell to a drug, drug candidate, toxin, pollutant etc.

In general, a method to predict a toxic effect of a compound or a composition of compounds comprises the steps of exposing a model animal or a cell culture to the compound or composition of compounds, detecting or measuring the differential expression (mRNA, protein-content, etc) of one or more genes from Table 3 in a biological sample of said model animal or said cell culture compared to a control, and comparing the determined differential expression to the differential expression disclosed in Table 3.

In the context of the present invention, the term “expression level” comprises, inter alia, the gene expression levels defined as RNA-levels, i.e. the amount or quality of RNA, mRNA, and the corresponding cDNA-levels; and the protein expression levels.

The term “differential gene expression” in accordance with this invention relates to the up- or down-regualtion of genes in tissues or cells derived from treated animals/cell cultures in comparison to control animals/cell cultures. These genes, which are differentially expressed, are also refered to as marker genes. Furthermore, it is envisaged that said comparison is carried out in a computer-assisted fashion. Said comparison may also comprise the analysis in high-throughput screens.

Most preferably, an increase or decrease of the expression level in (a) marker gene(s) as listed in Table 3 and as detected by the inventive method is indicative of hepatotoxic liability. It is also preferred that in addition to the said marker genes, the gene expression profile as depicted in Table 3 will also be analyzed in order to categorize hepatotoxic liability of the test compound(s).

It is also envisaged that the method of the invention comprises the comparison of differentially expressed marker genes, i.e. marker genes which are up or down-regulated in tissues, cells, body fluids etc, from biological samples after exposure to model compounds (as exemplified in Tables 1 and 2), with markers which are not changed, i.e. which are not diagnostic for hepatotoxicity. Such unchanged marker genes comprise, inter alia, the ribosomal RNA control as employed in the appended examples, as well as house-keeping genes (N° 10 as depicted in Table 4).

The detection and/or measurement of the expression levels of the genes from Table 3 according to the methods of the present invention may comprise the detection of an increase, decrease and/or the absence of a specific nucleic acid molecule, for example mRNA or cDNA.

Methods for the detection/measurement of mRNA and or cDNA levels are well known in the art and comprise methods as described in the appended examples, but are not limited to microarray- and PCR-technology.

In addition, protein expression levels from marker genes as listed in Table 4 and of some genes in Table 3 can also be assessed. Methods for the detection/measurement of protein levels are well known in the art and include, but are not limited to Western-blot, two-dimensional electrophoresis, ELISA, RIA, immunohistochemistry, etc.

Additional assay formats may be used to monitor the induced change of the expression level of a gene identified in Table 3. For instance, mRNA expression may be monitored directly by hybridization of probes to the nucleic acids of the invention. Cell lines are exposed to the agent to be tested under appropriate conditions and time and total RNA or mRNA is isolated by standard procedures such as those disclosed in Sambrook et al (Molecular Cloning: A Laboratory 30 Manual, 2nd Ed. Cold Spring Harbor Laboratory Press, 1989).

Any assay format to detect gene expression may be used. For example, traditional Northern blotting, dot or slot blot, nuclease protection, primer directed amplification, RT-PCR, semi- or quantitative PCR, branched-chain DNA and differential display methods may be used for detecting gene expression levels. Those methods are useful for some embodiments of the invention. In cases where smaller numbers of genes are detected, amplification-based assays may be most efficient. Methods and assays of the invention, however, may be most efficiently designed with hybridization-based methods for detecting the expression of a large number of genes. Any hybridization assay format may be used, including solution-based and solid support-based assay formats.

In another assay format, cell lines that contain reporter gene fusions between the open reading frame and/or the transcriptional regulatory regions of a gene in Table 3 and any assayable fusion partner may be prepared. Numerous assayable fusion partners are known and readily available including the firefly luciferase gene and the gene encoding chloramphenicol acetyltransferase (Alam et al. (1990) Anal. Biochem. 188, 245-254). Cell lines containing the reporter gene fusions are then exposed to the compound to be tested under appropriate conditions and time. Differential expression of the reporter gene between samples exposed to the compound and control samples identifies compounds which modulate the expression of the nucleic acid.

Preferably in the method of the present invention, the expression of at least one gene as listed in Table 3 is detected/measured. Yet, it is also envisaged that the expression of at least two, at least three, at least five, at least ten, at least twenty, at least thirty, at least forty, at least fifty, at least one hundred genes as listed in Table 3 are detected/measured. Moreover, it is envisaged that the expression of nearly all genes from Table 3 or of all genes from Table 3 is detected. It is furthermore envisaged that specific patterns of differentially expressed marker genes as depicted in Table 3 are detected, measured and/or compared.

The above mentioned animal model to be employed in the methods of the present invention and comprising and/or expressing a maker gene as defined herein is a non-human animal, preferably a mammal, most preferably mice, rats, sheep, calves, dogs, monkeys or apes. Most preferred are rodent models such as rats and mice. The animal model also comprises non-human transgenic animals, which preferably express at least one toxicity marker gene as disclosed in Table 3.

Yet it is also envisaged that non-human transgenic animals be produced which do not express marker genes as disclosed in Table 3 or which over-express said marker genes.

Transgenic non-human animals comprising and/or expressing the up-regulated marker genes of the present invention or, in contrast which comprise silenced or less efficient versions of down-regulated marker genes for hepatotoxicity, as well as cells derived thereof, are useful models for studying hepatotoxicity mechanisms.

Accordingly, said transgenic animal model may be transfected or transformed with the vector comprising a nucleic acid molecule coding for a marker gene as disclosed in Table 3. Said animal model may therefore be genetically modified with a nucleic acid molecule encoding such at marker gene or with a vector comprising such a nucleic acid molecule. The term “genetically modified” means that the animal model comprises in addition to its natural genome a nucleic acid molecule or vector as defined herein and coding for a toxicity marker of Table 3 or at least a fragment thereof. Said additional genetic material may be introduced into the animal model or into one of its predecessors/parents. The nucleic acid molecule or vector may be present in the genetically modified animal model or cell either as an independent molecule outside the genome, preferably as a molecule which is capable of replication, or it may be stably integrated into the genome of the animal model or cell thereof.

As mentioned herein above, the method of the present invention may also employ a cell culture. Preferred are cultures of primary animal cells or cell lines. Suitable animal cells are, for instance, primary mammalian hepatocytes; insect cells, vertebrate cells, preferably mammalian cell lines, such as e.g. CHO, HeLa, NIH3T3 or MOLT-4. Further suitable cell lines known in the art are obtainable from cell line depositories, like the American Type Culture Collection (ATCC). Most preferred are primary hepatocyte cultures or hepatic cell lines comprising rodent or human primary hepatocyte cultures including monolayer, sandwich cultures and slices cultures; as well as rodent cell lines such as BRL3, NRL clone9, and human cell lines such as HepG2 cells.

Cells or cell lines used in the method of the present invention may be transfected or transformed with a vector comprising a nucleic acid molecule coding for a marker gene as disclosed in Table 3. Said cell or cell line may therefore be genetically modified with a nucleic acid molecule encoding such a marker gene or with a vector comprising such a nucleic acid molecule. The term “genetically modified” means that the cell comprises in addition to its natural genome a nucleic acid molecule or vector as defined herein and coding for a toxicity marker of Table 3 or at least a fragment thereof. The nucleic acid molecule or vector may be present in the genetically modified cell either as an independent molecule outside the genome, preferably as a molecule which is capable of replication, or it may be stably integrated into the genome of the cell.

In accordance with the present invention, the term “biological sample” or “sample” as employed herein means a sample which comprises material wherein said differential expression of marker genes may be measured and may be obtained. “Samples” may be tissue samples derived from tissues of non-human animals, as well as cell samples, derived from cells of non-human animals or from cell cultures. For animal experimentation, biological samples comprise target organ tissues obtained after necropsy or biopsy and body fluids, such as blood or urine. For possible clinical use of the markers, particular preferred samples comprise body fluids, like blood, sera, plasma, urine, synovial fluid, spinal fluid, cerebrospinal fluid, semen or lymph, as well as body tissues obtained by biopsy. Particularly documented in the appended examples are rat liver tissues and primary hepatocyte cultures. Peripheral blood samples were also obtained to analyze circulating liver enzymes.

The cell population that is exposed to the compound or composition may be exposed in vitro or in vivo. For instance, cultured or freshly isolated hepatocytes, in particular rat hepatocytes, may be exposed to the compound under standard laboratory and cell culture conditions. In another assay format, in vivo exposure may be accomplished by administration of the compound to a living animal, for instance a laboratory rat. Procedures for designing and conducting toxicity tests in in vitro and in vivo systems are well known, and are described in many texts on the subject, such as Loomis et al. (Loomis's Esstentials of Toxicology, 4th Ed. Academic Press, New York, 1996; Echobichon, The Basics of Toxicity Testing, CRC Press, Boca Raton, 1992; Frazier, editor, In Vitro Toxicity Testing, Marcel Dekker, New York, 1992) and the like. In in vitro toxicity testing, two groups of test organisms are usually employed: One group serves as a control and the other group receives the test compound in a single dose (for acute toxicity tests) or a regimen of doses (for prolonged or chronic toxicity tests). Since in some cases, the extraction of tissue as called for in the methods of the invention requires sacrificing the test animal, both the control group and the group receiving the compound must be large enough to permit removal of animals for sampling tissues, if it is desired to observe the dynamics of gene expression through the duration of an experiment. In setting up a toxicity study, extensive guidance is provided in the literature for selecting the appropriate test organism for the compound being tested, route of administration, dose ranges, and the like. Water or physiological saline (0.9% NaCl in water) is the solute of choice for the test compound since these solvents permit administration by a variety of routes. When this is not possible because of solubility limitations, vegetable oils such as corn oil or organic solvents such as propylene glycol may be used.

A method of predicting the mechanism of toxicity of a compound comprising detecting the level of expression in a tissue or cell sample exposed to the compound of one or more genes from Table 3 is also provided, wherein differential expression of the genes in Table 3 is associated with a specific mechanism of toxicity.

By “mechanism of toxicity” it is meant the measurable manifestation of the toxic event, regarding target organ, time of onset, underlying molecular mechanism (i.e. DNA-damage, formation of protein adduct, etc) histopathological and biochemical findings such as circulating liver enzymes. Gene expression profiles can also be characteristic of a toxicity mechanism.

Different mechanisms of toxicity are known for hepatotoxins. Direct acting compounds are those compounds that cause damage to macromolecules, in particular proteins and lipids by directly interacting with them. This interaction could occur through the test compound itself or, more commonly, through a highly reactive metabolite thereof. Histological manifestations of these class of hepatoxicity include hepatocellular necrosis, lipid peroxidation and elevation of circulating levels of enzymes of hepatic origin such as ALT (alanine aminotransferase). Inflammation can also be observed due to the activation of the hepatic Kupffer cells. Steatotic compounds are those that cause an accummulation of fat in the liver. There are two types of steatosis: macrovesicular steatosis and microvesicular steatosis. All the test compounds used in this invention belong to the latter type. Characteristic of microvesicular steatosis is the accumulation of small lipid vesicles in the hepatocytes (so-called fatty liver), which usually lead to accute liver failure. The underlying molecular mechanisms are thought to be an inhibition of mitochondrial beta oxidation (due to mitochondrial damage) and/or an inhibition of the export of fatty acids from the hepatocyte. Compounds leading to cholestasis impair the bile flow, causing the clinical manifestation of jaundice. Intrahepatic cholestasis involves usually the inhibition of the bile acid transporters in the hepatocytes, leading to an accummulation of bile acids. Increased bile acids are responsible for slight hepatocyte injury, little inflammation and the elevation of circulating alkaline phosphatase (G. L. Plaa and W. R. Hewitt Ed. “Toxicology of the liver, 2^{nd} Ed., Target organ toxicology series, 1997; Fromenty and Pessayre (1995). Inhibition of mitochondrial beta-oxidation as a mechanism of hepatotoxicity. Pharmacol Ther 1, 101-54; Jaeschke, Gores, Cederbaum, Hinson, Pessayre and Lemasters (2002). Mechanisms of hepatotoxicity. Toxicol Sci 2, 166-76).

Detection of toxic potential as identified and/or obtained by the methods of the present invention are particularly useful in the development of new drugs in terms of safety.

Moreover, a method of predicting at least one toxic effect of a compound, comprising detecting the level of expression of progression elevated gene 3 (PEG-3) or Translocon associated protein (TRAP) from Table 4 in a tissue or cell sample exposed to the compound is provided, wherein differential expression of PEG-3 and TRAP is indicative of at least one toxic effect. The preferred toxic effect of the compound in the present method is hepatotoxicity.

PEG-3 belongs to the family of GADD-45 and GADD-153, which are genes up-regulated upon DNA-damage. While GADD-genes are known stress-inducible markers that lead to a cell cycle arrest (Seth A, Giunta S, Franceschil C, Kola I, Venanzoni M C (1999). Regulation of the human stress response gene GADD153 expression: role of ETS1 and FLI-1 gene products. Cell Death Differ 6(9), 902-7; Tchounwou P B, Wilson B A, Ishaque A B, Schneider J. Atrazine potentiation of arsenic trioxide-induced cytotoxicity and gene expression in human liver carcinoma cells (HepG2). Mol Cell Biochem. 222, 49-59; Tchounwou P B, Ishaque A B, Schneider J (2001). Cytotoxicity and transcriptional activation of stress genes in human liver carcinoma cells (HepG2) exposed to cadmium chloride. Mol Cell Biochem. 222, 21-8; Tchounwou P B, Wilson B A, Ishaque A B, Schneider J (2001). Transcriptional activation of stress genes and cytotoxicity in human liver carcinoma cells (HepG2) exposed to 2,4,6-trinitrotoluene, 2,4-dinitrotoluene, and 2,6-dinitrotoluene. Environ Toxicol. 16, 209-16.; Zhan Q, Fan S, Smith M L, Bae I, Yu K, Alamo I Jr, O'Connor P M, Formace A J Jr (1996). Abrogation of p53 function affects gadd gene responses to DNA base-damaging agents and starvation. DNA Cell Biol 15, 805-15), PEG-3 is involved in progression (Park J S, Qiao L, Su Z Z, Hinman D, Willoughby K, McKinstry R, Yacoub A, Duigou G J, Young C S, Grant S, Hagan M P, Ellis E, Fisher P B, Dent P (2001). Ionizing radiation modulates vascular endothelial growth factor (VEGF) expression through multiple mitogen activated protein kinase dependent pathways. Oncogene 20, 3266-80.; Su Z Z, Goldstein N I, Jiang H, Wang M N, Duigou G J, Young C S, Fisher P B (1999). PEG-3, a nontransforming cancer progression gene, is a positive regulator of cancer aggressiveness and angiogenesis. Proc Natl Acad Sci USA. 96, 15115-20; Su Z, Shi Y, Friedman R, Qiao L, McKinstry R, Hinman D, Dent P, Fisher P B (2001). PEA3 sites within the progression elevated gene-3 (PEG-3) promoter and mitogen-activated protein kinase contribute to differentialPEG-3 expression in Ha-ras and v-raf oncogene transformed rat embryo cells. Nucleic Acids Res 29, 1661-71; Su, Z. Z., Shi, Y., and Fisher, P. B. (1997). Subtraction hybridization identifies a transformation progression-associated gene PEG-3 with sequence homology to a growth arrest and DNA damage-inducible gene. Proc Natl Acad Sci USA 94, 9125-30). The results of the present invention show that the up-regulation of PEG-3 seems to be triggered earlier than that of GADDs, so that it is a possible early marker for cell damage.

TRAP proteins are part of a complex whose function is to bind Ca²⁺ to the membrane of the endoplasmic reticulum (ER) and regulate thereby the retention of ER resident proteins (Hartmann E, Gorlich D, Kostka S, Otto A, Kraft R, Knespel S, Burger E, Rapoport T A, Prehn S (1993). A tetrameric complex of membrane proteins in the endoplasmic reticulum. Eur J. Biochem. 214, 375-81).

Compounds used in the method of the present invention may be unknown compounds or compounds which are known to elicit a toxic effect in an organism.

Compounds in accordance with the method of the present invention include, inter alia, peptides, proteins, nucleic acids including DNA, RNA, RNAi, PNA, ribozymes, antibodies, small organic compounds, small molecules, ligands, and the like.

The compounds whose toxic effect is to be predicted with the method(s) of the present invention do not only comprise single, isolated compounds. It is also envisaged that mixtures of compounds are screened with the method of the present invention. It is also possible to employ natural products and extracts, like, inter alia, cellular extracts from prokaryotic or eukaryotic cells or organisms.

In addition, the compound identified by the inventive method as having low toxic effect can be employed as a lead compound to achieve modified site of action, spectrum of activity and/or organ specificity, and/or improved potency, and/or decreased toxicity (improved therapeutic index), and/or decreased side effects, and/or modified onset of therapeutic action, duration of effect, and/or modified pharmakinetic parameters (resorption, distribution, metabolism and excretion), and/or modified physico-chemical parameters (solubility, hygroscopicity, color, taste, odor, stability, state), and/or improved general specificity, organ/tissue specificity, and/or optimized application form and route, and may be modified by esterification of carboxyl groups, or esterification of hydroxyl groups with carbon acids, or esterification of hydroxyl groups to, e.g. phosphates, pyrophosphates or sulfates or hemi succinates, or formation of pharmaceutically acceptable salts, or formation of pharmaceutically acceptable complexes, or synthesis of pharmacologically active polymers, or introduction of hydrophylic moieties, or introduction/exchange of substituents on aromates or side chains, change of substituent pattern, or modification by introduction of isosteric or bioisosteric moieties, or synthesis of homologous compounds, or introduction of branched side chains, or conversion of alkyl substituents to cyclic analogues, or derivatisation of hydroxyl group to ketales, acetales, or N-acetylation to amides, phenylcarbamates, or synthesis of Mannich bases, imines, or transformation of ketones or aldehydes to Schiffs bases, oximes, acetates, ketales, enolesters, oxazolidines, thiozolidines or combinations thereof.

In another embodiment, the present invention provides for a set of nucleic acid primers, wherein the primers specifically amplify at least two of the genes from Table 3. The set of nucleic acid primers may also specifically amplify at least 5, at least 10, at least 20, at least 30 of the genes from Table 3. The set of nucleic acid primers may also specifically amplify nearly all or all of the genes from Table 3.

Moreover, the present invention provides for a set of nucleic acid probes, wherein the probes comprise sequences which hybridize to at least two of the genes from Table 3. The set of nucleic acid probes may comprise sequences which hybridize to at least 5, at least 10, at least 20, at least 30 of the genes from Table 3. The set of nucleic acid probes may also comprise sequences which hybridize to nearly all or all of the genes from Table 3.

In a further embodiment, the set of probes may be attached to a solid support. A solid support comprising at least two probes, wherein each of the probes comprises a sequence that specifically hybridizes to a gene in Table 3 is also provided. The solid support may also comprise at least 5 probes, at least 10, at least 20, at least 30 probes. The solid support may also comprise all or nearly all probes, wherein each of the probes comprises a sequence that specifically hybridizes to a gene in Table 3.

Solid supports containing oligonucleotide or cDNA probes for differentially expressed genes of the invention can be filters, polyvinyl chloride dishes, particles, beads, microparticles or silicon or glass based chips, etc. Such chips, wafers and hybridization methods are widely available, for example, those disclosed in WO95/11755. Any solid surface to which a nucleotide sequence can be bound, either directly or indirectly, either covalently or non-covalently, can be used. A preferred solid support is a DNA chip. These contain a particular probe in a predetermined location on the chip. Each predetermined location may contain more than one molecule of the probe, but each molecule within the predetermined location has an identical sequence. Such predetermined locations are termed features. There may be, for example, from 2, 10, 100, 1000 to 10000, 100000 or 400000 of such features on a single solid support. The solid support, or the area within which the probes are attached may be on the order of about a square centimeter.

Probes corresponding to the genes of Table 3 may be attached to single or multiple solid support structures, e.g., the probes may be attached to a single chip or to multiple chips to comprise a chip set. Probe arrays for expression monitoring can be made and used according to any techniques known in the art (see for example, Lockhart et al., Nat. Biotechnol. (1996) 14, 1675-1680; McGall et al., Proc. Nat. Acad. Sci. USA (1996) 93, 13555-60). Such probe arrays may contain at least two or more probes that are complementary to or hybridize to two or more of the genes described in Table 3. For instance, such arrays may contain probes that are complementary or hybridize to at least 2, 3, 4, 5, 6, 7, 8, 9, 10, 20, 30, 50, 70, 100 or more of the genes described herein. Preferred arrays contain probes for all or nearly all of the genes listed in Table 3. In a preferred embodiment, arrays are constructed that contain probes to detect all or nearly all of the genes of Table 3 on a single solid support substrate, such as a chip. The sequences of the expression marker genes of Table 3 are available in public databases and their GenBank Accession Number is provided (see www.rzcbi.nlm.nih.gov/). These sequences may be used in the methods of the invention or may be used to produce the probes and arrays of the invention. As described above, in addition to the sequences of the GenBank Accessions Numbers disclosed in Table 3, sequences such as naturally occurring variant or polymorphic sequences may be used in the methods and compositions of the invention. For instance, expression levels of various allelic or homologous forms of a gene disclosed in the Table 3 may be assayed. Any and all nucleotide variations that do not alter the functional activity of a gene listed in Table 3, including all naturally occurring allelic variants of the genes herein disclosed, may be used in the methods and to make the compositions (e.g., arrays) of the invention.

Probes based on the sequences of the genes described above may be prepared by any commonly available method. “Probe” refers to a hybridizable nucleotide sequence that can be attached to a solid support or used in a liquid form. As used herein a “probe” is defined as a nucleic acid sequence, capable of binding to a target nucleic acid of complementary sequence through one or more types of chemical bonds, usually through complementary base pairing, usually through hydrogen bond formation. As used herein, a probe may include natural (i.e., A, G, U, C, or T) or modified bases (7-deazaguanosine, inosine, etc.). In addition, the bases in probes may be joined by a linkage other than a phosphodiester bond, so long as it does not interfere with hybridization. Thus, probes may be peptide nucleic acids in which the constituent bases are joined by peptide bonds rather than phosphodiester linkages. Said probes are specific oligonucleotides or cDNA-fragments. Oligonucleotide probes or cDNAs for screening or assaying a tissue or cell sample are preferably of sufficient length to specifically hybridize only to appropriate, complementary genes or transcripts. Typically the oligonucleotide probes will be at least 10, 12, 14, 16, 18, 20 or 25 nucleotides in length. In some cases, longer probes of at least 30, 40, or 50 nucleotides will be desirable. Typically, the cDNA probes will be between 300 and 1000 nucleotides in length. As used herein, oligonucleotide sequences that are complementary to one or more of the genes described in Table 3 refer to probes that are capable of hybridizing under stringent conditions to at least part of the nucleotide sequences of said genes. Such hybridizable probes will typically exhibit at least about 75% sequence identity at the nucleotide level to said genes, preferably about 80% or 85% sequence identity or more preferably about 90% or 95% or more sequence identity to said genes. The phrase “hybridizing specifically to” refers to the binding, duplexing, or hybridizing of a molecule substantially to or only to a particular nucleotide sequence or sequences under stringent conditions when that sequence is present in a complex mixture (e.g., total cellular) DNA or RNA. Assays and methods of the invention may utilize available formats to simultaneously screen at least 2, preferably about tens to thousends different nucleic acid hybridizations. The terms “background” or “background signal intensity” refer to hybridization signals resulting from non-specific binding, or other interactions, between the labeled target nucleic acids and components of the oligonucleotide array (e.g., the probes, control probes, the array substrate, etc.). Background signals may also be produced by intrinsic fluorescence of the array components themselves. A single background signal can be calculated for the entire array, or a different background signal may be calculated for each target nucleic acid. One of skill in the art will appreciate that where the probes to a particular gene hybridize well and thus appear to be specifically binding to a target sequence, they should not be used in a background signal calculation. Background can also be calculated as the average signal intensity produced by regions of the array that lack any probes at all.

One of skill in the art will appreciate that an enormous number of array designs are suitable for the practice of this invention. The array will typically include a number of test probes, at least 2, preferably tens to thousends that specifically hybridize to the sequences of interest. Probes may be produced from any region of the identified genes. In instances where the gene reference in the Tables is an EST, probes may be designed from that sequence or from other regions of the corresponding full-length transcript that may be available in any of the sequence databases, such as those herein described. Any available software may be used to produce specific probe sequences, including, for instance, software available from Applied Biosystems (Primer Express). The said probes may be attached to the solid support by a variety of methods, including among others synthesis onto the glass and spotting of a specified amount of cDNA onto the support. In addition to test probes that bind the target nucleic acid(s) of interest, the arrays can contain a number of control probes. The control probes may fall into three categories referred to herein as 1) normalization controls; 2) expression level controls; and 3) unspecific binding controls. Normalization controls are probes that are complementary to labeled reference oligonucleotides or other nucleic acid sequences that are added to the nucleic acid sample to be screened. The signals obtained from the normalization controls after hybridization provide a control for variations in hybridization conditions, label intensity, “reading” efficiency and other factors that may cause the signal of a perfect hybridization to vary between arrays. Signals read from all other probes in the array may be divided by the signal (e.g., fluorescence intensity) from the control probes thereby normalizing the measurements. Virtually any probe may serve as a normalization control. However, it is recognized that hybridization efficiency varies with base composition and probe length. Preferred normalization probes are selected to reflect the average length of the other probes present in the array. Expression level controls are probes that hybridize specifically with constitutively expressed genes in the biological sample. Virtually any constitutively expressed gene provides a suitable target for expression level controls. Typically expression level control probes have sequences complementary to subsequences of constitutively expressed “housekeeping genes” including, but not limited to the actin gene, the transferrin receptor gene, the GAPDH gene, and the like. Unspecific binding controls can be but are not limited to DNA from other species (i.e. Hering Sperm DNA) that should not hybridize with the target sequences or mismatched sequences. Mismatched sequences are oligonucleotide probes or other nucleic acid probes identical to their corresponding test or control probes except for the presence of one or more mismatched bases. A mismatched base is a base selected so that it is not complementary to the corresponding base in the target sequence to which the probe would otherwise specifically hybridize. One or more mismatches are selected such that under appropriate hybridization conditions (e.g. stringent conditions) the test or control probe would be expected to hybridize with its target sequence, but the mismatch probe would not hybridize (or would hybridize to a significantly lesser extent). Unspecific binding controls thus provide a control for non-specific binding or cross hybridization to a nucleic acid in the sample other than the target to which the probe is directed.

Cell or tissue samples may be exposed to the test compound in vitro or in vivo. When cultured cells or tissues are used, appropriate mammalian liver extracts may also be added with the test agent to evaluate compound s that may require biotransformation to exhibit toxicity. In a preferred format, primary isolates of animal or human hepatocytes which already express the appropriate complement of drug-metabolizing enzymes may be exposed to the test compound without the addition of mammalian liver extracts. The genes which are assayed according to the present invention are typically in the form of mRNA or reverse transcribed mRNA. The genes may be cloned or not. The genes may be amplified or not. The cloning and/or amplification do not appear to bias the representation of genes within a population. In some assays, it may be preferable, however, to use polyA+ RNA as a source, as it can be used with less processing steps. As is apparent to one of, ordinary skill in the art, nucleic acid samples used in the methods and assays of the invention may be prepared by any available method or process. Methods of isolating total mRNA are well known to those of skill in the art. For example, methods of isolation and purification of nucleic acids are described in detail in Chapter 3 of Laboratory Techniques in Biochemistry and Molecular Biology: Hybridization With Nucleic Acid Probes, Part I Theory and Nucleic Acid Preparation, P. Tijssen, Ed., Elsevier, N.Y. (1993). Such samples include RNA samples, but also include cDNA synthesized from a mRNA sample isolated from a cell or tissue of interest. Such samples also include DNA amplified from the cDNA, and RNA transcribed from the amplified DNA. One of skill in the art would appreciate that it is desirable to inhibit or destroy RNase present in homogenates before homogenates are used. Biological samples may be of any biological tissue or fluid or cells from any organism as well as cells raised in vitro, such as cell lines and tissue culture cells. Frequently the sample will be a tissue or cell sample that has been exposed to a compound, agent, drug, pharmaceutical composition, potential environmental pollutant or other composition, In some formats, the sample will be a “clinical sample” which is a sample derived from a patient. Typical clinical samples include, but are not limited to, sputum, blood, blood-cells (e.g. white cells), tissue or fine needle biopsy samples, urine, peritoneal fluid, and pleural fluid, or cells therefrom. Biological samples may also include sections of tissues, such as frozen sections or formalin fixed sections taken for histological purposes.

Nucleic acid hybridization simply involves contacting a probe and target nucleic acid under conditions where the probe and its complementary target can form stable hybrid duplexes through complementary base pairing (See WO99/32660). The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids. The term “stringent conditions” refers to conditions under which a probe will hybridize to its target sequence, but with only insubstantial hybridization to other sequences. Stringent conditions are sequence-dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH. Stringent conditions may also be achieved with the addition of destabilizing agents such as formamide. Under low stringency conditions (e.g. low temperature and/or high salt) hybrid duplexes (e.g. DNA:DNA, RNA:RNA, or RNA:DNA) will form even where the annealed sequences are not perfectly complementary. Thus, specificity of hybridization is reduced at lower stringency. Conversely, at higher stringency (e.g. higher temperature or lower salt) successful hybridization tolerates fewer mismatches. One of skill in the art will appreciate that hybridization conditions may be selected to provide any degree of stringency. In a preferred embodiment, hybridization is performed at low stringency, to ensure hybridization and then subsequent washes are performed at higher stringency to eliminate mismatched hybrid duplexes. Successive washes may be performed at increasingly higher stringency until a desired level of hybridization specificity is obtained. Stringency can also be increased by addition of agents such as formamide. Hybridization specificity may be evaluated by comparison of hybridization to the test probes with hybridization to the various controls that can be present (e.g., expression level control, normalization control, mismatch controls, etc.). In general, there is a tradeoff between hybridization specificity (stringency) and signal intensity. Thus, in a preferred embodiment, the wash is performed at the highest stringency that produces consistent results and that provides a signal intensity greater than approximately 10% of the background intensity. Thus, in a preferred embodiment, the hybridized array may be washed at successively higher stringency solutions and read between each wash. Analysis of the data sets thus produced will reveal a wash stringency above which the hybridization pattern is not appreciably altered and which provides adequate signal for the particular probes of interest.

The hybridized nucleic acids are typically detected by detecting one or more labels attached to the sample nucleic acids. The labels may be incorporated by any of a number of means well known to those of skill in the art (see WO99/32660).

The present invention includes databases containing DNA sequence information as well as gene expression information from tissue or cells exposed to various standard toxins, such as those herein described (see Tables 1-2). The Toxicogenomics database is supported by in-house developed software (RACE-R, F. Hoffmann-La Roche A G, Basle, Switzerland) which allows the storage, analysis and comparison of absolute (intensity) and relative (fold-induction) gene expression data obtained by a variety of methods such as the aforementioned Affymetrix high density arrays, low density spotted arrays, PCR, etc. This database allows also for the incorporation of additional data such as sample description, biochemical parameters, histological evaluation, etc. Additional databases may also contain information associated with a given DNA sequence or tissue sample such as descriptive information about the gene associated with the sequence information (see Table 3), or descriptive information concerning the clinical status of the tissue sample, or the animal from which the sample was derived. The database may allow the use of algorithms (i.e. Toxicology Model Matcher, F. Hoffmann-La Roche A G, Basle, Switzerland) for the extensive comparison of gene expression profiles between known or unknown test compounds and compounds which are already in the database as listed in Tables 1 and 2. Methods for the configuration and construction of such databases are widely available, for instance, see U.S. Pat. No. 5,953,727. The databases of the invention may be linked to an outside or external database such as GenBank (www.ncbi.nlm.nih.gov/entrez.index.html); KEGG 25 (www.genome.adjp/kegg); SPAD (www.grt.kyushu-u.acjp/spad/index.html); HUGO (www.gene.ucl.ac.uk/hugo); Swiss-Prot (www.expasy.ch.sprot); Prosite (www.expasy.ch/tools/scnpsitl.h&l); OMIM (www.ncbi.nlm.nih.gov/omim); GDB (www.gdb.org); and GeneCard (bioinformatics.weizmann.ac.il/cards). In a preferred embodiment, as described in Tables 3, 7 and 8, the external database is GenBank and the associated databases maintained by the National Center for Biotechnology Information (NCBI) (www.ncbi.nlm.nih.gov). Any appropriate computer platform may be used to perform the necessary comparisons between sequence information, gene expression information and any other information in the database or information provided as an input. For example, a large number of computer workstations are available from a variety of manufacturers. Client/server environments, database servers and networks are also widely available and appropriate platforms for the databases of the invention. The databases of the invention may be used to determine the cell type or tissue in which a given gene is expressed and to allow determination of the abundance or expression level of a given gene in a particular tissue or cell. The databases of the invention may also be used to present information identifying the expression level in a tissue or cell of a set of genes comprising one or more of the genes in Table 3, comprising the step of comparing the expression level of at least one gene in Table 3 in a cell or tissue exposed to a test compound to the level of expression of the gene in the database. Such methods may be used to predict the toxic potential of a given compound by comparing the level of expression of a gene or genes in Table 3 from a tissue or cell sample exposed to the test compound to the expression levels found in a control tissue or cell samples exposed to a standard toxin or hepatotoxin such as those herein described.

The gene expression data generated by the methods of the present invention may be analysed by various methods known in the art, including but not limited to hierarchical clustering, self-organizing maps and support vector machines. Support Vector Machines (SVMs), a class of supervised learning algorithms originally introduced by Vapnik and co-workers, have already been shown to perform well in multiple areas of biological analysis (Boser, B. E., Guyon, I. M., Vapnik, V. N. (1992) A training algorithm for optimal margin classifiers. In Proceedings of the 4^{th} Annual International Conference on Computational Learning Theory, ACM Press, Pittsburgh, Pa., 144-152; Vapnik, V. N. (1998) Statistical Learning Theory. Wiley, New York; Scholkopf, B., Guyon, I. M., Weston, J. (2002) Statistical Learning and Kernel Methods in Bioinformatics. In Proceedings NATO Advanced Studies Inst. on Artificial Intelligence and Heuristics Methods for Bioinformatics, San Miniato, Italy October 1-11).

Given a set of training examples, SVMs are able to recognize informative patterns in the input data and generalize on previously unseen data. Trivial solutions, which overfit the training data, are avoided by minimizing the bound on the expected generalization error. In contrast to unsupervised methods like hierarchical clustering and self-organizing maps, the SVM approach takes advantage of prior knowledge in the form of class labels attached to the training examples. The extraordinary robustness with respect to sparse and noisy data makes SVMs the tool of choice in a growing number of applications. They are particularly well suited to analyze microarray expression data because of their ability to handle situations where the number of features (genes) is very large compared to the number of training patterns (chip replicates). It has been demonstrated in several studies that SVMs typically tend to outperform other classification techniques in this field (Brown, M. P. S., Grundy, W. N., Lin, D., Cristianini, N., Sugnet, C. W., Furey, T. S., Ares, M., Haussler, D. (2000) Knowledge-based analysis of microarray gene expression data by using support vector machines. PNAS 97, 262-267; Furey, T. S., Cristianini, N., Duffy, N., Bednarski, D. W., Schummer, M., Haussler, D. (2000) Support Vector machine classification and validation of cancer tissue samples using microarray expression data. Bioinformatics 16, 906-914; Yeang, C., Ramaswamy, S., Tamayo, P., Mukherjee, S., Rifkin, R. M., Angelo, M., Reich, M., Lander, E., Mesirov, J., Golub, T. (2001) Molecular classification of multiple tumor types. Bioinformatics 17, 316-322). In addition, the method proved effective in discovering informative features such as genes which are especially relevant for the classification and therefore might be critically important for the biological processes under investigation (Guyon, I. M., Weston, J., Barnhill, S., Vapnik, V. N. (2002) Gene Selection for Cancer Classification using Support Vector Machines. Machine Learning 46, 389-442).

The SVM approach can be used to generate classifiers for discrimination of a specific toxicant class from all other classes, but also to generate discriminators to distinguish between a specific toxicant and controls can be defined. Alternatively classifiers for discrimination of toxic and non-toxic compounds can be constructed. These classifiers are useful to predict toxicity as well as for identification of a specific toxicity mechanism.

Recursive feature elimination (RFE) allows identifying genes that contribute to the greatest extent to classification. In each iteration, a certain fraction of genes is removed from the training procedure, selected by the corresponding weights in the decision function. The least important genes are omitted for the next iteration. During this process, quality parameters of the resulting SVM classifiers are monitored. The final choice of a best subset of genes is made on the basis of classification accuracy, model simplicity and gene count. This method makes no orthogonality assumptions about gene expression levels but implicitly takes into account correlation between the single gene expression measurements. It results in a minimized set of predictive genes by effectively removing noise and redundancy from the set of all genes on the chip. The support vector mechanism, where borderline (and not ‘typical’) training patterns play a crucial role in classifications, was shown to assist in the feature elimination process by preventing genes that are irrelevant for classification but nevertheless differentially expressed in the majority of chip samples from gaining predominant influence (Guyon, I. M., Weston, J., Barnhill, S., Vapnik, V. N. (2002) Gene Selection for Cancer Classification using Support Vector Machines. Machine Learning 46, 389-442). Using RFE a small subset of genes is selected. This subset can subsequently be used as a diagnostic biomarker set to predict toxicity and/or the mechanism of toxicity.

The present invention therefore also provides a computer system comprising a database containing DNA sequence information and expression information of at least two of the genes from Table 3 from tissue or cells exposed to a hepatotoxin, and a user interface.

The invention further includes kits combining, in different combinations, nucleic acid primers for the amplification of the genes of Table 3, solid supports with attached probes, reagents for use with the solid supports, protein reagents encoded by the genes of Table 3, signal detection and array-processing instruments, gene expression databases and analysis and database management software described above. The kits may be used, for example, to predict or model the toxic response of a test compound, to monitor the progression of hepatic disease states, to identify genes that show promise as new drug targets and to screen known and newly designed drugs as discussed above.

The databases packaged with the kits are a compilation of expression patterns from human or laboratory animal genes and gene fragments (corresponding to the genes of Table 3). In particular, the database software and packaged information include the expression results of Table 3 that can be used to predict toxicity of a test compound. In another format, database and software information may be provided in a remote electronic format, such as a website, the address of which may be packaged in the kit.

The invention is now described by reference to the following examples and figures which are merely illustrative and are not to be construed as a limitation of scope of the present invention.

### EXAMPLES

Commercially available reagents referred to in the examples were used according to manufacturer's instructions unless otherwise indicated.

### Example 1

### Hepatotoxicity Assay with Non-Human Animals

All animals received human care as specified by Swiss law and in accordance with the “Guide for the care and use of laboratory animals” published by the NIH. Male Wistar rats (generally 5 animals/dose-group) were purchased from BRL (Füllingsdorf, Switzerland) and housed individually. Treated animals were dosed either orally, intraperitoneally, or intravenously with several doses of test compounds (Table 1). The test compounds were categorized according to their toxic manifestation in the rat liver. Control animals received the same volume of vehicle as placebo. Necropsy was performed 6 or 24 hours after a single administration and liver samples from the left medial lobe were placed immediately in RNALater (Ambion, Tex., USA) for RNA extraction and gene expression analysis. Samples in RNALater were stored at −20° C. until further processing. Additional liver samples were snap-frozen in liquid nitrogen for measurement of intrahepatic lipids and/or proteins.

### Example 2

### Hepatocyte Cell Culture Assay Toxicity

Hepatocytes were isolated from adult male Wistar rats by two-step collagenase liver perfusion previously described (Goldlin C. R., Boelsterli U. A. (1991). Reactive oxygen species and non-peroxidative mechanisms of cocaine-induced cytotoxicity in rat hepatocyte cultures. Toxicology 69, 79-91). Briefly, the rats were anaesthetized with sodium pentobarbital (120 mg/kg, i.p.). The perfusate tubing was inserted via the portal vein, then the v. cava caudalis was cut, and the perfusion was started. The liver was first perfused for 5 min with a preperfusing solution consisting of calcium-free, EGTA (0.5 mM)-supplemented, HEPES (20 mM)-buffered Hank's balanced salt solution (5.36 mM KCl, 0.44 mM KH₂PO₄, 137 mM NaCl, 4.2 mM NaHCO₃, 0.34 mM Na₂HPO₄, 5.55 mM D-glucose). This was followed by a 12-min perfusion with NaHCO₃ (25 mM)-supplemented Hank's solution containing bovine CaCl₂ (5 mM), and collagenase (0.2 U/ml). Flow rate was maintained at 28 ml/min and all solutions were kept at 37° C. After in situ perfusion the liver was excised and the liver capsule was mechanically disrupted. The cells were suspended in William's Medium E without phenol red (WME, Sigma Chemie, Buchs, Switzerland) and filtered through a set of tissue sieves (30-, 50-, and 80-mesh). Dead cells were removed by a sedimentation step (1×g, for 15 min at 4° C.) followed by a Percoll (Sigma) centrifugation step and an additional centrifugation in WME (50 g, 3 min). Hepatocyte viability was assessed by trypan blue exclusion and typically lied between 85% and 95%. The cells were seeded into collagen-coated 6-well Falcon Primaria® plates at a density of 9×10⁵ cells/well in 2 ml WME supplemented with 10% fetal calf serum (BioConcept, Allschwil, Switzerland), penicillin (100 U/ml, Sigma Chemie, Buchs, Switzerland), streptomycin (0.1 mg/ml, Sigma Chemie, Buchs, Switzerland), insulin (100 nM, Sigma Chemie, Buchs, Switzerland), and dexamethasone (100 nM). After an attachment period of 3 hrs, the medium was replaced by 1.5 ml/well serum-free WME, supplemented with antibiotics and hormones, and incubated overnight at 37° C. in an atmosphere of 5% CO2/95% air. Cells were then incubated with the test compounds or vehicle (Table 2) and harvested for RNA extraction at 6 or 24 hours.

### Example 3

### Measurement of Circulating and Hepatic Enzymes

In the hepatotoxicity assay with non-human animals as described in Example 1, circulating enzymes of hepatic origin, as well as the hepatic lipid content were assessed. Blood samples for clinical chemistry were obtained shortly before sacrifice. The enzymatic activities of aspartate aminotransferase (AST), alanine aminotransferase (ALT), lactate dehydrogenase (LDH) and 5-nucleotidase (5-ND) were measured in serum samples. Liver lipids were extracted using liver homogenates as described by Freneaux et al (Freneaux, E., Labbe, G., Letteron, P., The Le, D., Degott, C., Geneve, J., Larrey, D., and Pessayre, D. (1988). Inhibition of the mitochondrial oxidation of fatty acids by tetracycline in mice and in man: possible role in microvesicular steatosis induced by this antibiotic. Hepatology 8, 1056-62) and the contents of triglycerides, phospholipids and total lipids were measured. Automated analysis was performed using commercially available test kits (Roche Diagnostics, Mannheim, Germany) on a Cobas Fara autoanalyzer (Roche, Basel, Switzerland).

### Example 4

### RNA Sample Preparation

RNA isolation from hepatocytes was typically performed by resuspending approximately 3 Mio. Cells/1.2 mL RNAzol (Tel-Test Inc., TX, USA). For RNA isolation from liver tissue, a portion of tissue of approximately 100 mg was transferred to a tube containing 1.2 ml RNAzol. Cells or tissue in RNAzol were disrupted in FastPrep tubes for 20 seconds in a Savant homogenizer (Bio101, Buena Vista, Calif., U.S.A.). Total RNA was isolated according to the manufacturer's instruction and quantified by measuring the optical density at 280 nm. The quality of RNA was assessed with gel electrophoresis.

### Example 5

### Synthesis and Hybridization of cRNA

Double stranded cDNA was synthesized from 20 μg of total RNA using a cDNA Synthesis System (Roche Diagnostics, Mannheim, Germany) with the oligo(dT)₂₄ T7prom)₆₅ primer. The MEGAScript T7 kit (Ambion, Austin, Tex., U.S.A.) was used to transcribe the cDNA into cRNA in the presence of Biotin-11-CTP and Biotin-16-UTP (Enzo, Farmingdale, N.Y., U.S.A.) according to the instructions supplied with the kit. After purification with the RNeasy kit (Qiagen, Hilden, Germany) integrity of the cRNA was checked using gel electrophoresis. 10-15 μg fragmented cRNA were used for hybridization to the RG-U34A array (Affymetrix GeneChip® array, Santa Clara, Calif.). The oligonucleotide array used in the present study contains probe sets for over 5000 rat genes. Hybridization and staining were performed basically as described previously (Lockhart D J, Dong H, Byrne M C, Follettie M T, Gallo M V, Chee M S, Mittmann M, Wang C, Kobayashi M, Horton H, Brown E L (1996). Expression monitoring by hybridization to high-density oligonucleotide arrays. Nat Biotechnot. 14, 1675-80.; de Saizieu A, Certa U, Warrington J, Gray C, Keck W, Mous J. (1998). Bacterial transcript imaging by hybridization of total RNA to oligonucleotide arrays. Nat Biotechnol. 16, 45-8). Arrays were scanned with a confocal laser scanner (Hewlett-Packard, Palo Alto, Calif., USA).

### Example 6

### Expression Analysis

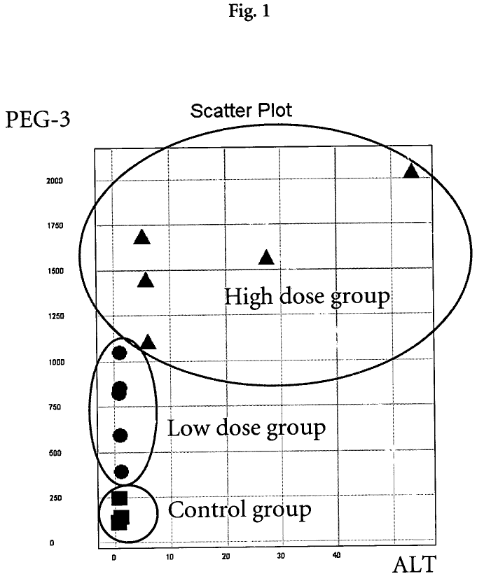

After hybridization and scanning, the expression level of each gene was calculated by subtracting the fluorescence intensity of the mismatch probes from the match signal (=average difference) using the GENECHIP 3.1 software or its up-dated versions MAS 4.0 and MAS 5.0 (Affymetrix). Gene expression data were further analyzed with an in-house-developed data analysis tool (RACE-A, Hoffmann-La Roche, Basel, Switzerland). Data sets of treated versus time and vehicle matched control were generated for each treatment (treated vs. controls) and compared. Analyzed data were stored in a toxicogenomics database (RACE-R, Hoffmann-La Roche, Basel, Switzerland). For the gene expression analysis, difference of means, fold-induction, statistical significance (Students' t-test) were applied for querying the data base. Likewise, increase in circulating liver enzymes (ALT, AST, ALP, etc.) were analyzed. A linear regression was performed between gene expression levels for each gene and circulating enzyme levels. An example of correlation between gene expression and circulating ALT levels is given in FIG. 1.

### Example 7

### Detection of Profiles and Specific Marker Genes

Gene expression changes common to a number of compounds belonging to the same hepatotoxicity mechanism were considered profiles typical for this mechanism. The profiles were determined after the in vivo exposure of adult male Wistar rats to 18 compounds, from which 6 were steatotic, 6 were direct acting and 6 were cholestatic. For each tested compound, one or several independent experiments were performed. The results are depicted in Table 3, showing that 680 differentially expressed genes were identified in vivo: 30 of them were only regulated in livers after exposure to steatotic compounds; 18 were regulated after exposure to cholestatic compounds; and 559 after exposure to direct acting compounds. 11 genes showed regulation by all types of tested toxic compounds and are probably related to cellular stress. Others show regulation by two types of toxicity mechanisms.

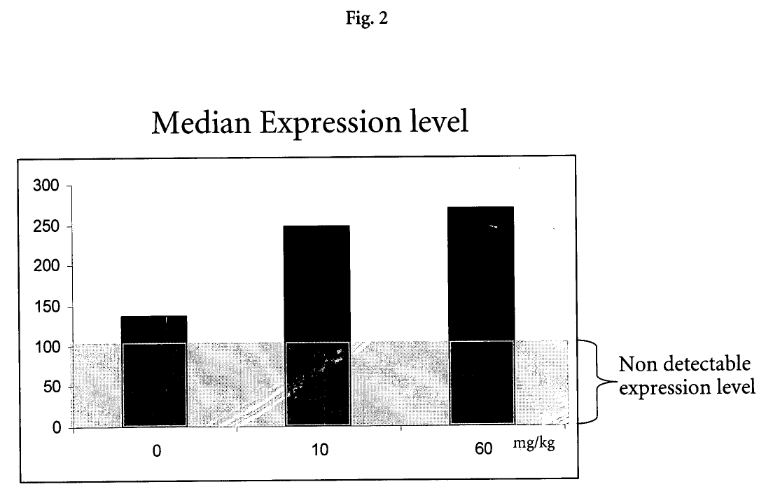

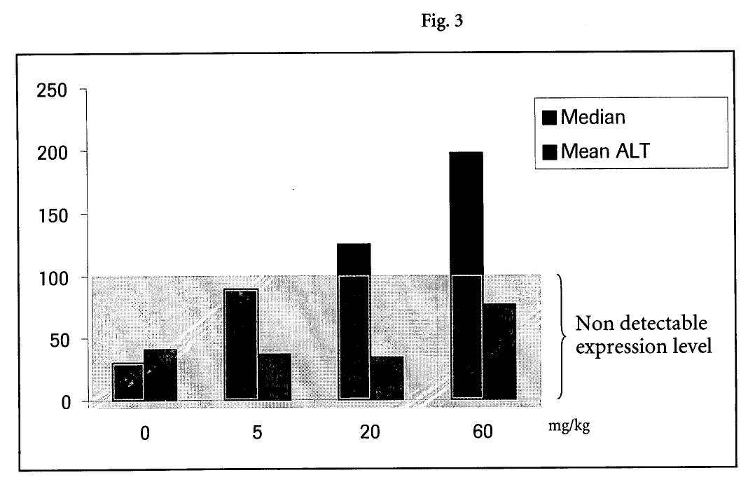

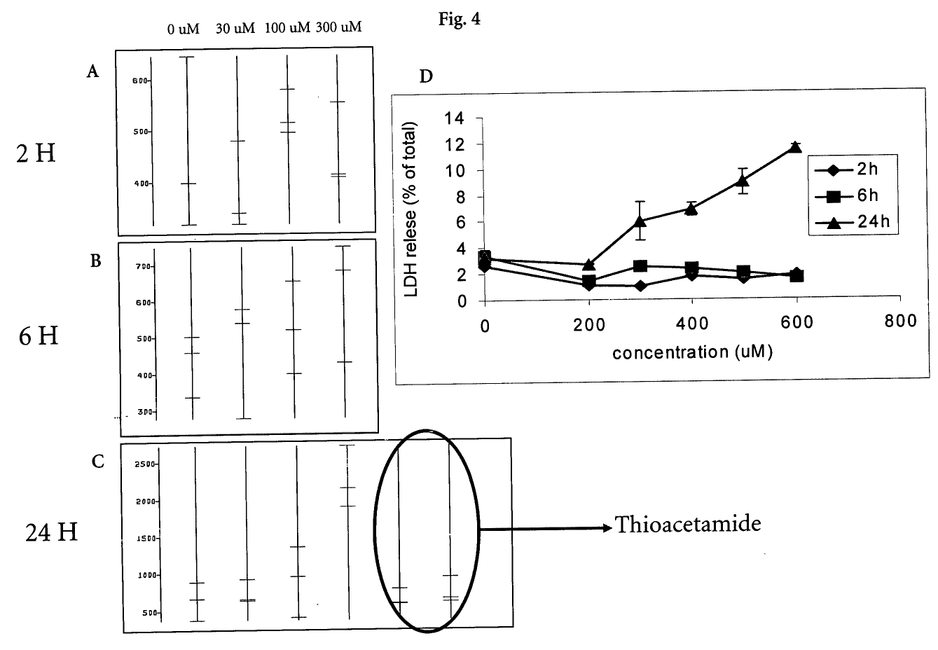

In addition to the defined profiles, gene expression levels and their correlation (linear regression) with the circulating liver enzymes were used to select genes whose expression varied across the samples. These genes were chosen as possible toxicity markers and selected with less stringent filtering criteria. Candidate marker genes were categorized as follows:
a) differentially expressed genes (up-regulated in animals showing elevated enzymes in comparison to the matched controls, 2-fold change, p<0.05), b) differentially expressed genes (down-regulated in animals showing elevated enzymes in comparison to the matched controls, 2-fold change, p<0.05); c) genes that fulfilled the criteria in a) but that additionally showed an up-regulation at doses and/or time points at which no elevation of circulating enzymes could be detected; d) genes that fulfilled the criteria in b) but that additionally showed an down-regulation at doses and/or time points at which no elevation of circulating enzymes could be detected. Some of these marker genes are listed in Table 4. Among them, PEG-3 (progression elevated gene 3, # 1 in the said Table 4) and TRAP (translocon-associated protein, #9 in Table 4) showed very good characteristics as a possible early predictor in vivo (Michael Fountoulakis, Maria-Cristina de Vera, Flavio Crameri, Franziska Boess, Rodolfo Gasser, Silvio Albertini, Laura Suter: “Modulation of gene and protein expression by carbon tetrachloride in the rat liver”, Toxicol and Appl. Pharmacol. 2002 Aug. 15; 183(1):71-80) as well as in vitro. This is represented in FIGS. 1 through 4.

### Example 8

### Data Validation for Selected Genes

The regulation of the mRNA levels of several candidate genes (Table 4) was verified with quantitative RT-PCR. Specific primers for these genes have been designed in order to evaluate the expression with RT-PCR using SybrGreen assay (Table 6). For each performed RT-PCR reaction, the specificity of the assay was evaluated with dissociation curve software (Applied Biosystems), as well as by assessment of the size of the product using either gel electrophoresis or Agilent Bioanalyzer. The obtained results are described in this example and generally confirmed the results obtained using GeneChips (analysis performed with microarray suite version MAS 4.0 or MAS 5.0).

Western-blot analysis was performed on 10 micrograms total liver protein following standard laboratory procedures. Proteins transferred onto a nitrocellulose membrane were incubated with the first antibody (Anti-cytochrome p450 2B1, raised in goat, purchased at GenTest, Massachusetts); followed by an incubation with the second antibody (donkey anti-sheep/Goat Immunoglobulin Horseradish Peroxidase Conjugated, from Chemikon). Chemoluminiscence was quantified by densitometry in a Multimage Light Cabinet (Alpha Inotech Corporation, San Leandro, Calif., USA) using Lumilight (Roche Diagnostics AG, Rotkreuz, Switzerland) solution.

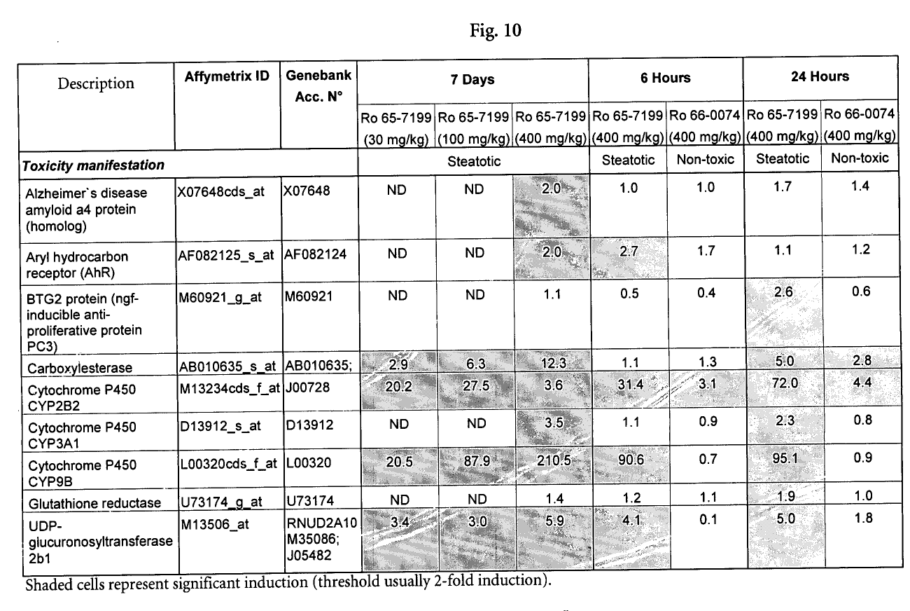

Real-time PCR is a truly quantitative method, while genechip-analysis is only semi-quantitative for transcriptional expression studies. In a first step, the induction of the mRNA levels of 9 candidate genes (Table 5 and FIG. 10) was verified with quantitative PCR. The results obtained with both methods showed that the expression of these genes would allow the differentiation of a steatotic compound from a pharmacological analogue that does not show steatotic potential. These genes could be possible diagnostic and predictive molecular markers for different mechanisms of hepatic toxicity. As an internal control, the house-keeping gene glycernine-aldehyde-phosphate-dehydrogenase (GAPDH) was also analyzed.

5-HT6 Receptor Antagonists

RT-PCR results confirmed that the two 5-HT6 receptor antagonists could be distinguished using the expression levels of few marker genes (FIG. 10). Note that for some of the selected genes, a slight induction with the non-toxic compound Ro 66-0074 was also observed. However, this induction was minor when compared to the larger effect elicited by the toxic compound Ro 65-7199 so that differentiation of both compounds remains possible.

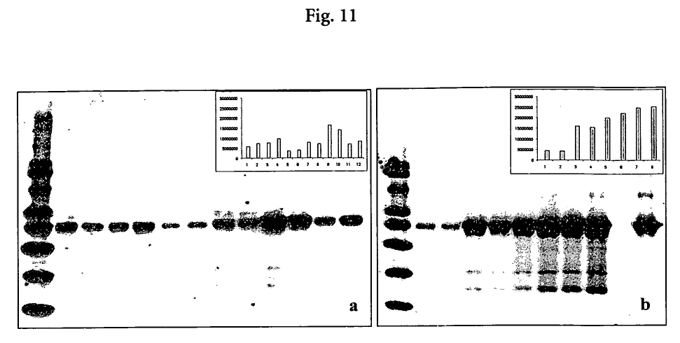

In addition, Western blots were performed using specific antibodies against the cytochrome P450 CYP2B family in order to evaluate if the clear induction of messenger RNA also led to an increase in the hepatic protein levels. The results of the protein levels of CYP2B closely paralleled the amounts of mRNA at 24 hours and at 7 days after Ro 65-7199 treatment. However, the induction of CYP2B could not be detected 6 hours after administration of Ro 65-7199, in spite of the increased levels of messenger RNA. This is due to the time lag between protein and mRNA induction (FIG. 11).

Direct Acting Compounds Regulate the GADD-Family

Further experiments with RT-PCR confirmed results regarding the induction of genes from the GADD family, namely GADD-45, GADD-153 and PEG-3 by direct acting compounds (Hydrazine, Thioacetamide, 1,2-dichlorobenzene) (Table 9). The induction of these genes correlates with the histopathological findings in a time related fashion: While PEG-3 seems to be an early marker, GADD-45 and GADD-143 appear regulated at later time points, when the tissue damage is obvious by conventional endpoints. These results are in line with literature and confirm the assumption that PEG-3 is an early marker for hepatic damage.

Induction of EGR1 by Tolcapone (Tasmar) and Dinitrophenol

Tolcapone (Tasmar) is a human hepatotoxin with no known toxicity in the rat. In this experiment, a slight induction of EGR1 was detected after exposure of rats to a high dose of Tolcapone (300 mg/kg) and dinitrophenol (10 and 30 mg/kg). The induction was slight and showed high inter-individual variability but experiments with RT-PCR confirmed these results (Table 10).

EGR1 (early growth response gene 1, EMBL_ro:rnngfla) is a transcription factor that is also known by synonyms such as Zif268, NGF1-A, Krox24, TIS8. Its name derives of the kinetics of its induction, since it is a primary transcribed signal: the protein can be induced within minutes of a stimulus and then decays within hours (Khachigian, L. M. and T. Collins, Inducible expression of Egr-1-dependent genes. A paradigm of transcriptional activation in vascular endothelium. Circ Res, 1997. 81(4): p. 457-61; Yan, S. F., et al., Egr-1: is it always immediate and early? J Clin Invest, 2000. 105(5): p. 553-4). Nevertheless, maintained high expression of EGR1 has been described in atherosclerotic tissue and in connection to cell death in Alzheimer's disease, establishing a relationship between EGR1 overexpression and chronic conditions (McCaffrey, T. A., et al., High-level expression of Egr-1 and Egr-1-inducible genes in mouse and human atherosclerosis. J Clin Invest, 2000. 105(5): p. 653-62). Its function under normal conditions is still unclear, since EGR1-null mice display normal phenotype with exception of infertility in females (Lee, S. L., et al., Luteinizing hormone deficiency and female infertility in mice lacking the transcription factor NGFI-A (Egr-1). Science, 1996. 273(5279): p. 1219-21). Thus, the physiological role of EGR1 might only become manifest upon environmental challenge. This gene has been found overexpressed in several pathologic conditions, including exposure to ionising radiation, prostate cancer and hypoxia (Weichselbaum, R. R., et al., Radiation-induced tumour necrosis factor-alpha expression: clinical application of transcriptional and physical targeting of gene therapy. Lancet Oncol, 2002. 3(11): p. 665-71). The up-regulation of EGR1 after hypoxia leads to vascular and perivascular tissue damage. In particular in lung, EGR1 induction leads an increase of Tissue Factor (TF) and to deposition of fibrin in the lung vasculature (Yan, S. F., et al., Egr-1, a master switch coordinating upregulation of divergent gene families underlying ischemic stress. Nat Med, 2000. 6(12): p. 1355-61). EGR1-deficient mice show significantly reduced prostate tumor formation and significantly less pulmonary vascular permeability and therefore better survival after ischemic injury (Abdulkadir, S. A., et al., Impaired prostate tumorigenesis in Egr1-deficient mice. Nat Med, 2001. 7(1): p. 101-7, Ten, V. S. and D. J. Pinsky, Endothelial response to hypoxia: physiologic adaptation and pathologic dysfunction. Curr Opin Crit Care, 2002. 8(3): p. 242-50).

The literature reports suggest a possible involvement of EGR1 as an early signal to injury that triggers subsequent tissue damage. In particular in the liver, a link between mitochondrial uncoupling (as caused by dinitrophenol) and induction of EGR1 was established. Also, tolcapone (Tasmar) has been described as having mitochondrial uncoupling properties in vitro. Thus, it is suggested that the induction of EGR1 in rats exposed to tolcapone (Tasmar) might lead to hepatic tissue injury if the physiological environment (i.e. existing disease or genetic background) is appropriate. This would explain the low incidence of human hepatotoxicity caused by tolcapone (Tasmar).

### Example 9

### Expression Data Analysis with Support Vector Machines

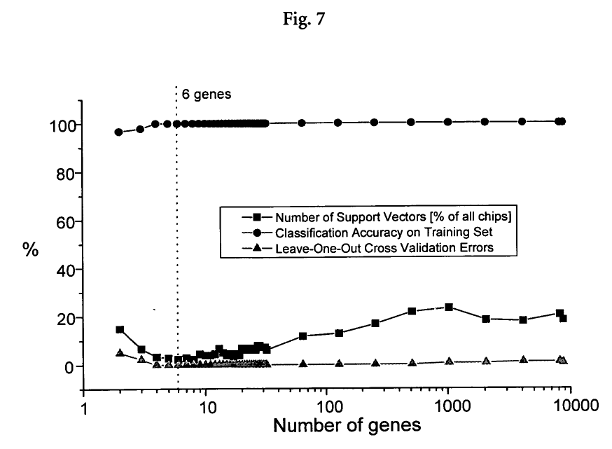

Adult male Wistar rats were dosed in vivo and the resulting expression profile in liver was determined. SVMs were built for the discrimination between 3 different classes of hepatotoxicants, non-toxic substances and controls. The training set consisted of 180 gene expression profiles from individual animals treated with direct acting, cholestatic, steatotic, non-toxic compounds and corresponding vehicle-dosed controls. As a first step the chips were rescaled to a median value of 0 and a standard deviation of 1. Subsequently chips were presented to a linear kernel SVM (for classifier training the SVM software package from William Stafford Noble, Department of Computer Science, Columbia University, New York was used. Procedures for the Recursive Feature Elimination (RFE), automation of the whole training cycles and further data analysis were developed in house, using the PERL language.) Single binary classifiers for each category of chips were obtained by training one group against all others (‘One-vs-All’ training method). Multi-class classification for a given test chip was then carried out by combining the outputs of all binary classifiers. A leave-one-out cross validation procedure was applied to assess the quality of the trained machines with respect to the training data. This method consists of removing one sample from the training set, building a classifier on the basis of the remaining data and then testing on the withheld example. By removing all replicates of one compound from the training data, followed by classifying these chips with the resulting decision function, the individual contribution of the given compound for a successful classifier could be examined. RFE was used to investigate the relationship between the number of genes for generating the classifiers, the resulting prediction accuracy, cross-validation errors and the number of used support vectors. The first iteration reduced the gene count to a multiple of 2. In each subsequent iteration the gene count was halved until 32 genes remained. Afterwards only one gene per iteration was removed. An example of a RFE for the direct acting class is shown in FIG. 7.

Based on classification accuracy, model simplicity and gene count a SVM was selected for each class. As can be seen the SVM for discrimination of direct acting compounds from all others was based on 6 genes. The corresponding gene numbers for the other SVMs were:

Steatotic (6 genes), cholestatic (21 genes), non-toxic (19 genes) and controls (46 genes).

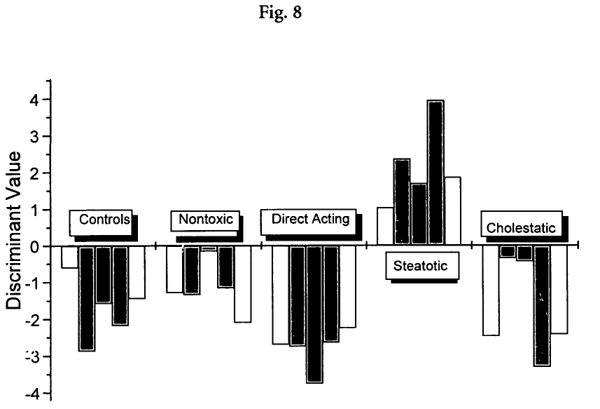

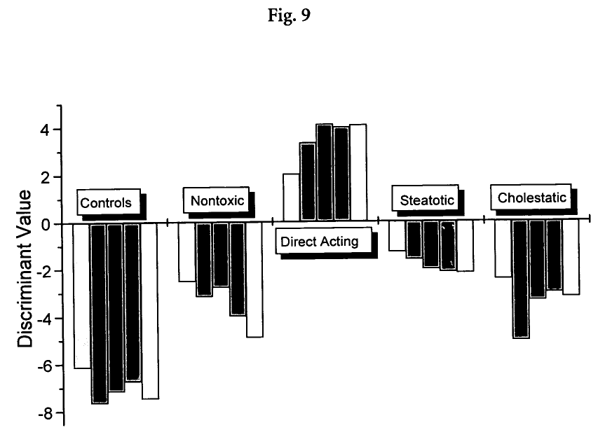

Compounds not present in the initial training set were subsequently classified based on their expression profiles. Expression profiles for individual animals were classified using the 5 previously generated support vector machines. The successful identification of amineptine as a steatotic compound is depicted in FIG. 8 and the identification of 1,2-Dichlorobenzene as a direct acting compound is shown in FIG. 9. The bigger a positive value is, the better is the data fit into a specific class defined by the respective SVM. A negative discriminant value means that data do not fit into a compound class.

The above-described method was used to find class-specific genes that allow discrimination of a class from all other classes (class-discriminating genes, Table 7).

The same approach was also used to find toxicant specific genes for each of the categories. Using RFE SVMs for the discrimination of direct acting compounds from controls were generated. Based on classification accuracy, model simplicity and gene count one SVM was selected for discrimination of the direct acting group from the control group. This classifier was based on 14 genes (specific genes for the direct acting group, Table 8).

The same procedure was repeated for the steatotic and the cholestatic class. The classifier for the cholestatic group contained 34 genes and the classifier for the steatotic group contained 3 genes (Table 8). The genes required to separate a class from all other classes or just from controls can therefore be different.

TABLE 1

| | | | Target | Hepatotoxicity |
|---|---|---|---|---|
| Compound | Dose levels | | organ | Mechanism |
| Chlorpromazine | 15 | mg/kg | Liver | Cholestatic |
| Cyclosporine A | 5, 15 and 30 | mg/kg | Liver | Cholestatic |
| Erythromycin | 734 | mg/kg | Liver | Cholestatic |
| Glibenclamide | 2.5 and 25 | mg/kg | Liver | Cholestatic |
| Lithocholic acid | 60 and 120 | μmol/kg | Liver | Cholestatic |
| Ro 48-5695 (ETA) | 25 | mg/kg | Liver | Cholestatic |
| Dexamethasone | 0.6 | mg/kg | Liver | Cyp inducer/prolif |
| 1,2-Dichlorobenzene | 1.5 and 4.5 | mmol/kg | Liver | Direct Acting |
| Aflatoxin B1 | 1 and 4 | mg/kg | Liver | Direct Acting |
| Bromobenzene | 1 and 3 | mmol/kg | Liver | Direct Acting |
| Carbon tetrachloride | 0.25 and 2 | ml/kg | Liver | Direct Acting |
| Diclofenac | 10, 30, 100 | mg/kg | Liver | Direct Acting |
| Hydrazine | 10, 60, 90 | mg/kg | Liver | Direct Acting |
| Nitrofurantoin | 5, 20, 60 | mg/kg | Liver | Direct Acting |
| Thioacetamide | 2, 10, 50 | mg/kg | Liver | Direct Acting |
| Concanavaline A | 0.1, 20 | mg/kg | Liver | Hepatitis/Infammation |
| Tacrine | 5, 15 and 35 | mg/kg | Liver | Human Hepatotox |
| Tempium | 20 and 1000 | mg/kg | Liver | Human hepatotox |
| (Lazabemide) | | | | |
| Tolcapone (Tasmar) | 300 | mg/kg | Liver | Human hepatotox |
| 1,4-Dichlorobenzene | 4.5 | mmol/kg | Liver | Non toxic |
| Amineptin | 125, 250, 500 | μmol/kg | Liver | Steatotic |
| Amiodarone | 50, 100, 600 | mg/kg | Liver | Steatotic |
| Doxycycline | 5, 20, 40 | mg/kg | Liver | Steatotic |
| Ro 28-1674 (GKA) | 250 | mg/kg | Liver | Steatotic |
| Ro 28-1675 (GKA) | 100 | mg/kg | Liver | Steatotic |
| Ro 65-7199 (5HT6) | 30, 100, 400 | mg/kg | Liver | Steatotic |
| Tetracycline | 125, 200, 250 | μmol/kg | Liver | Steatotic |
| Dinitrophenol | 10 and 30 | mg/kg | Liver | Uncoupling |

Ro 48-5695: Pyridin-2-ylcarbamic acid 2-[6-(5-isopropyl-pyridin-2-ylsulfonylamino)-5-(2-methoxy-phenoxy)-2-morpholin-4-yl-pyrimidin-4-yloxyl-ethyl ester;
Ro 28-1674: 3-Cyclopentyl-2-[S]-4-(methanesulfonyl-phenyl)-N-thiazol-2-yl-propionamide;
Ro 28-1675: 3-Cyclopentyl-2[R]-(4-methanesulfonyl-phenyl)-N-thiazol-2-yl-propionamide.

TABLE 2

| | | | Hepatocyte culture | Hepatotoxicity |
|---|---|---|---|---|
| Compound | Test Concentrations | | System | Mechanism |
| Chlorpromazine | 10, 30 100 | μM | Monolayer | Cholestatic |
| Cyclosporine A | 0.5 and 5 | μM | Monolayer | Cholestatic |
| Erythromycin | 100 and 300 | μM | Monolayer | Cholestatic |
| Lithocholic acid | 10 and 30 | μM | Monolayer | Cholestatic |
| Ro 48-5695 (ETA) | 20 and 60 | μM | Monolayer | Cholestatic |
| Cyproterone Acetate | 5 and 25 | μM | Monolayer | Cyp inducer/prolif |
| Phenobarbital | 200 and 2000 | μM | Monolayer | Cyp inducer/prolif |
| Clofibrate | 100 and 1000 | μM | Monolayer | Cyp inducer/prolif |
| Acetaminophen | 1000, 2500 and 5000 | μM | Monolayer | Direct Acting |
| Acetaminophen | 1000, 2500 | μM | Sandwich | Direct Acting |
| Bromobenzene | 1000 and 2000 | μM | Monolayer | Direct Acting |
| Carbon tetrachloride | 3000 and 5000 | μM | Monolayer | Direct Acting |
| Hydrazine | 8000 and 16000 | μM | Monolayer | Direct Acting |
| Methapyrilene | 20 | μM | Sandwich | Direct Acting |
| Methapyrilene | 100, 300 and 1000 | μM | Monolayer | Direct Acting |
| Nitrofurantoin | 20, 100 and 200 | μM | Monolayer | Direct Acting |
| Thioacetamide | 3000 and 10000 | μM | Monolayer | Direct Acting |
| Thioacetamide-S- | 30, 100 and 300 | μM | Monolayer | Direct Acting |
| Oxide | | | | |
| Amineptin | 500, 1000 and 1500 | μM | Monolayer | Steatotic |
| Amiodarone | 30, 70, 100 and 300 | μM | Monolayer | Steatotic |
| Doxycycline | 100, 500 and 1000 | μM | Monolayer | Steatotic |
| Perhexiline | 3, 10 and 30 | μM | Monolayer | Steatotic |
| Ro 28-1674 (GKA) | 19 and 75 | μM | Monolayer | Steatotic |
| Ro 28-1675 (GKA) | 19 and 75 | μM | Monolayer | Steatotic |
| Ro 65-7199 (5HT) | 20 and 100 | μM | Monolayer | Steatotic |
| Tetracycline | 100 and 500 | μM | Monolayer | Steatotic |

TABLE 3
Gene identifiers are given as the Affymetrix GeneChip ®
RG-U34A. The accession numbers refer to GenBank and for each type of
hepatotoxicity, the direction of the gene regulation is indicated (1 for up-regulation, 1-
for down-regulation). Blank cells indicate the lack of regulation under the used analysis
criteria.

| | | Direct | | | Acc | SEQ |
|---|---|---|---|---|---|---|
| Affymetrix ID | Cholestatic | Acting | Steatotic | Profile | Number | ID NO |
| AF023087_s_at | 1 | 1 | 1 | Unspecific | AF023087 | 1 |
| D11445exon#1- | 1 | 1 | 1 | Unspecific | D11445 | 2 |
| 4_s_at | | | | | | |
| L25785_at | −1 | −1 | −1 | Unspecific | L25785 | 3 |
| M18416_at | 1 | 1 | 1 | Unspecific | M18416 | 4 |
| M58634_at | 1 | 1 | 1 | Unspecific | M58634 | 5 |
| M60921_g_at | 1 | 1 | 1 | Unspecific | M60921 | 6 |
| rc_AA891041_at | 1 | 1 | 1 | Unspecific | AA891041 | 7 |
| rc_AA893485_at | −1 | −1 | −1 | Unspecific | AA893485 | 8 |
| rc_AI137856_s_at | 1 | 1 | 1 | Unspecific | AI137856 | 9 |
| rc_AI172293_at | −1 | −1 | −1 | Unspecific | AI172293 | 10 |
| U75397UTR#1_s_at | 1 | 1 | 1 | Unspecific | U75397 | 11 |
| AF003835_at | | −1 | 1 | Steatotic/ | AF003835 | 12 |
| | | | | Direct Acting | | |
| AF014503_at | | 1 | 1 | Steatotic/ | AF014503 | 13 |
| | | | | Direct Acting | | |
| AF079864_at | | −1 | −1 | Steatotic/ | AF079864 | 14 |
| | | | | Direct Acting | | |
| D14989_f_at | | −1 | −1 | Steatotic/ | D14989 | 15 |
| | | | | Direct Acting | | |
| D17370_at | | −1 | −1 | Steatotic/ | D17370 | 16 |
| | | | | Direct Acting | | |
| D17370_g_at | | −1 | −1 | Steatotic/ | D17370 | 17 |
| | | | | Direct Acting | | |
| D44495_s_at | | 1 | 1 | Steatotic/ | D44495 | 18 |
| | | | | Direct Acting | | |
| E01524cds_s_at | | 1 | 1 | Steatotic/ | E01524 | 19 |
| | | | | Direct Acting | | |
| J02585_at | | −1 | −1 | Steatotic/ | J02585 | 20 |
| | | | | Direct Acting | | |
| L16764_s_at | | 1 | 1 | Steatotic/ | L16764 | 21 |
| | | | | Direct Acting | | |
| L16995_at | | −1 | −1 | Steatotic/ | L16995 | 22 |
| | | | | Direct Acting | | |
| M15481_at | | −1 | −1 | Steatotic/ | M15481 | 23 |
| | | | | Direct Acting | | |
| M21208mRNA_s_at | | 1 | 1 | Steatotic/ | M21208 | 24 |
| | | | | Direct Acting | | |
| M23572_at | | −1 | −1 | Steatotic/ | M23572 | 25 |
| | | | | Direct Acting | | |
| rc_AA799766_at | | 1 | 1 | Steatotic/ | AA799766 | 26 |
| | | | | Direct Acting | | |
| rc_AA800224_at | | 1 | −1 | Steatotic/ | AA800224 | 27 |
| | | | | Direct Acting | | |
| rc_AA891713_at | | 1 | 1 | Steatotic/ | AA891713 | 28 |
| | | | | Direct Acting | | |
| rc_AA892775_at | | 1 | 1 | Steatotic/ | AA892775 | 29 |
| | | | | Direct Acting | | |
| rc_AA946503_at | | 1 | 1 | Steatotic/ | AA946503 | 30 |
| | | | | Direct Acting | | |
| rc_AI145931_at | | −1 | −1 | Steatotic/ | AI145931 | 31 |
| | | | | Direct Acting | | |
| rc_AI169327_g_at | | 1 | 1 | Steatotic/ | AI169327 | 32 |
| | | | | Direct Acting | | |
| rc_AI176546_at | | 1 | 1 | Steatotic/ | AI176546 | 33 |
| | | | | Direct Acting | | |
| rc_AI177004_s_at | | −1 | −1 | Steatotic/ | AI177004 | 34 |
| | | | | Direct Acting | | |
| rc_AI639391_at | | −1 | −1 | Steatotic/ | AI639391 | 35 |
| | | | | Direct Acting | | |
| X05684_at | | −1 | −1 | Steatotic/ | X05684 | 36 |
| | | | | Direct Acting | | |
| X52625_at | | −1 | −1 | Steatotic/ | X52625 | 37 |
| | | | | Direct Acting | | |
| X91234_at | | −1 | −1 | Steatotic/ | X91234 | 38 |
| | | | | Direct Acting | | |
| AA848218_at | | | 1 | Steatotic | AA848218 | 39 |
| AB010635_s_at | | | 1 | Steatotic | AB010635 | 40 |
| AF022136_at | | | −1 | Steatotic | AF022136 | 41 |
| AF087839mRNA#1_s_at | | | 1 | Steatotic | AF087839 | 42 |
| K02814_at | | | 1 | Steatotic | K02814 | 43 |
| L09647_at | | | −1 | Steatotic | L09647 | 44 |
| L32132_at | | | 1 | Steatotic | L32132 | 45 |
| L36460mRNA_at | | | 1 | Steatotic | L36460 | 46 |
| M10068mRNA_s_at | | | 1 | Steatotic | M10068 | 47 |
| M14369exon#2_at | | | 1 | Steatotic | M14369 | 48 |
| M23566exon_s_at | | | 1 | Steatotic | M23566 | 49 |
| M35300_f_at | | | 1 | Steatotic | M35300 | 50 |
| rc_AA892522_at | | | −1 | Steatotic | AA892522 | 51 |
| rc_AA894316_at | | | −1 | Steatotic | AA894316 | 52 |
| rc_AA900582_at | | | 1 | Steatotic | AA900582 | 53 |
| rc_AI044985_at | | | −1 | Steatotic | AI044985 | 54 |
| rc_AI175764_s_at | | | −1 | Steatotic | AI175764 | 55 |
| rc_AI176351_s_at | | | 1 | Steatotic | AI176351 | 56 |
| rc_AI230256_at | | | −1 | Steatotic | AI230256 | 57 |
| rc_AI639108_at | | | −1 | Steatotic | AI639108 | 58 |
| rc_H31144_g_at | | | 1 | Steatotic | H31144 | 59 |
| S81478_s_at | | | −1 | Steatotic | S81478 | 60 |
| U02553cds_s_at | | | −1 | Steatotic | U02553 | 61 |
| U08214_s_at | | | 1 | Steatotic | U08214 | 62 |
| U35345_s_at | | | 1 | Steatotic | U35345 | 63 |
| U48220_at | | | −1 | Steatotic | U48220 | 64 |
| U88630_at | | | 1 | Steatotic | U88630 | 65 |
| X07648cds_g_at | | | 1 | Steatotic | X07648 | 66 |
| X62952_at | | | 1 | Steatotic | X62952 | 67 |
| X91810_at | | | 1 | Steatotic | X91810 | 68 |
| AA799276_at | | 1 | | Direct Acting | AA799276 | 69 |
| AB002086_g_at | | 1 | | Direct Acting | AB002086 | 70 |
| AB004096_at | | −1 | | Direct Acting | AB004096 | 71 |
| AB009636_at | | −1 | | Direct Acting | AB009636 | 72 |
| AB010466_s_at | | 1 | | Direct Acting | AB010466 | 73 |
| AB010963_s_at | | −1 | | Direct Acting | AB010963 | 74 |
| AB012230_g_at | | −1 | | Direct Acting | AB012230 | 75 |
| AB014722_g_at | | 1 | | Direct Acting | AB014722 | 76 |
| AB015433_s_at | | 1 | | Direct Acting | AB015433 | 77 |
| AB016536_s_at | | 1 | | Direct Acting | AB016536 | 78 |
| AB017188_at | | 1 | | Direct Acting | AB017188 | 79 |
| AB020504_at | | −1 | | Direct Acting | AB020504 | 80 |
| AF001417_s_at | | 1 | | Direct Acting | AF001417 | 81 |
| AF013144_at | | 1 | | Direct Acting | AF013144 | 82 |
| AF017637_at | | −1 | | Direct Acting | AF017637 | 83 |
| AF020618_at | | 1 | | Direct Acting | AF020618 | 84 |
| AF021935_at | | 1 | | Direct Acting | AF021935 | 85 |
| AF025308_f_at | | 1 | | Direct Acting | AF025308 | 86 |
| AF029240_g_at | | −1 | | Direct Acting | AF029240 | 87 |
| AF029310_at | | 1 | | Direct Acting | AF029310 | 88 |
| AF030086UTR#1_at | | −1 | | Direct Acting | AF030086 | 89 |
| AF030087UTR#1_at | | 1 | | Direct Acting | AF030087 | 90 |
| AF030087UTR#1_g_at | | 1 | | Direct Acting | AF030087 | 91 |
| AF036335_at | | 1 | | Direct Acting | AF036335 | 92 |
| AF037072_at | | −1 | | Direct Acting | AF037072 | 93 |
| AF039890mRNA_s_at | | −1 | | Direct Acting | AF039890 | 94 |
| AF041066_at | | −1 | | Direct Acting | AF041066 | 95 |
| AF044574_at | | −1 | | Direct Acting | AF044574 | 96 |
| AF045464_s_at | | 1 | | Direct Acting | AF045464 | 97 |
| AF050661UTR#1_at | | 1 | | Direct Acting | AF050661 | 98 |
| AF054618_s_at | | 1 | | Direct Acting | AF054618 | 99 |
| AF058791_at | | 1 | | Direct Acting | AF058791 | 100 |
| AF061443_at | | −1 | | Direct Acting | AF061443 | 101 |
| AF062594_g_at | | 1 | | Direct Acting | AF062594 | 102 |
| AF062741_g_at | | −1 | | Direct Acting | AF062741 | 103 |
| AF063447_at | | 1 | | Direct Acting | AF063447 | 104 |
| AF067650_at | | 1 | | Direct Acting | AF067650 | 105 |
| AF069782_at | | 1 | | Direct Acting | AF069782 | 106 |
| AF080507_at | | −1 | | Direct Acting | AF080507 | 107 |
| AF080507_g_at | | −1 | | Direct Acting | AF080507 | 108 |
| AF082124_s_at | | 1 | | Direct Acting | AF082124 | 109 |
| AF084186_s_at | | 1 | | Direct Acting | AF084186 | 110 |
| AF087037_at | | 1 | | Direct Acting | AF087037 | 111 |
| AJ011607_g_at | | −1 | | Direct Acting | AJ011607 | 112 |
| AJ012603UTR#1_at | | 1 | | Direct Acting | AJ012603 | 113 |
| AJ222724_at | | 1 | | Direct Acting | AJ222724 | 114 |
| AJ224120_at | | 1 | | Direct Acting | AJ224120 | 115 |
| D00636cds_s_at | | −1 | | Direct Acting | D00636 | 116 |
| D00636Poly_A_Site#1_s_at | | −1 | | Direct Acting | D00636 | 117 |
| D00698_s_at | | −1 | | Direct Acting | D00698 | 118 |
| D10354_s_at | | 1 | | Direct Acting | D10354 | 119 |
| D10587_g_at | | 1 | | Direct Acting | D10587 | 120 |
| D10756_g_at | | 1 | | Direct Acting | D10756 | 121 |
| D12769_at | | 1 | | Direct Acting | D12769 | 122 |
| D13122_f_at | | 1 | | Direct Acting | D13122 | 123 |
| D13623_at | | 1 | | Direct Acting | D13623 | 124 |
| D13623_g_at | | 1 | | Direct Acting | D13623 | 125 |
| D13667cds_s_at | | 1 | | Direct Acting | D13667 | 126 |
| D13907_at | | 1 | | Direct Acting | D13907 | 127 |
| D13978_s_at | | 1 | | Direct Acting | D13978 | 128 |
| D14014_at | | 1 | | Direct Acting | D14014 | 129 |
| D14425_s_at | | 1 | | Direct Acting | D14425 | 130 |
| D14564cds_s_at | | −1 | | Direct Acting | D14564 | 131 |
| D14987_f_at | | −1 | | Direct Acting | D14987 | 132 |
| D21800_g_at | | 1 | | Direct Acting | D21800 | 133 |
| D25224_at | | 1 | | Direct Acting | D25224 | 134 |
| D25224_g_at | | 1 | | Direct Acting | D25224 | 135 |
| D26564_at | | 1 | | Direct Acting | D26564 | 136 |
| D28557_s_at | | 1 | | Direct Acting | D28557 | 137 |
| D28560_at | | −1 | | Direct Acting | D28560 | 138 |
| D28560_g_at | | −1 | | Direct Acting | D28560 | 139 |
| D30649mRNA_s_at | | −1 | | Direct Acting | D30649 | 140 |
| D30666_at | | −1 | | Direct Acting | D30666 | 141 |
| D30804_at | | 1 | | Direct Acting | D30804 | 142 |
| D30804_g_at | | 1 | | Direct Acting | D30804 | 143 |
| D31662exon#4_s_at | | −1 | | Direct Acting | D31662 | 144 |
| D31874_at | | 1 | | Direct Acting | D31874 | 145 |
| D38061exon_s_at | | 1 | | Direct Acting | D38061 | 146 |
| D38062exon_s_at | | 1 | | Direct Acting | D38062 | 147 |
| D38381_s_at | | −1 | | Direct Acting | D38381 | 148 |
| D38468_s_at | | 1 | | Direct Acting | D38468 | 149 |
| D43964_at | | −1 | | Direct Acting | D43964 | 150 |
| D45247_at | | 1 | | Direct Acting | D45247 | 151 |
| D50694_at | | 1 | | Direct Acting | D50694 | 152 |
| D63704_at | | −1 | | Direct Acting | D63704 | 153 |
| D63704_g_at | | −1 | | Direct Acting | D63704 | 154 |
| D82928_at | | 1 | | Direct Acting | D82928 | 155 |
| D85435_at | | 1 | | Direct Acting | D85435 | 156 |
| D85435_g_at | | 1 | | Direct Acting | D85435 | 157 |
| D87839_g_at | | −1 | | Direct Acting | D87839 | 158 |
| D87991_at | | 1 | | Direct Acting | D87991 | 159 |
| D88034_at | | 1 | | Direct Acting | D88034 | 160 |
| D88890_at | | 1 | | Direct Acting | D88890 | 161 |
| D89069_f_at | | 1 | | Direct Acting | D89069 | 162 |
| D89514_at | | 1 | | Direct Acting | D89514 | 163 |
| D89983_at | | 1 | | Direct Acting | D89983 | 164 |
| D90109_at | | −1 | | Direct Acting | D90109 | 165 |
| D90265_s_at | | 1 | | Direct Acting | D90265 | 166 |
| E12286cds_at | | −1 | | Direct Acting | E12286 | 167 |
| E12625cds_at | | −1 | | Direct Acting | E12625 | 168 |
| J02589mRNA#2_at | | −1 | | Direct Acting | J02589 | 169 |
| J02646_at | | 1 | | Direct Acting | J02646 | 170 |
| J02679_s_at | | 1 | | Direct Acting | J02679 | 171 |
| J02962_at | | 1 | | Direct Acting | J02962 | 172 |
| J03179_g_at | | 1 | | Direct Acting | J03179 | 173 |
| J03572_i_at | | 1 | | Direct Acting | J03572 | 174 |
| J03969_at | | 1 | | Direct Acting | J03969 | 175 |
| J04187_at | | −1 | | Direct Acting | J04187 | 176 |
| J04791_s_at | | 1 | | Direct Acting | J04791 | 177 |
| J04943_at | | 1 | | Direct Acting | J04943 | 178 |
| J05035_g_at | | −1 | | Direct Acting | J05035 | 179 |
| J05122_at | | 1 | | Direct Acting | J05122 | 180 |
| J05166_at | | 1 | | Direct Acting | J05166 | 181 |
| J05210_at | | −1 | | Direct Acting | J05210 | 182 |
| J05210_g_at | | −1 | | Direct Acting | J05210 | 183 |
| K01934mRNA#2_at | | −1 | | Direct Acting | K01934 | 184 |
| K03045cds_r_at | | 1 | | Direct Acting | K03045 | 185 |
| K03249_at | | −1 | | Direct Acting | K03249 | 186 |
| L01267_at | | 1 | | Direct Acting | L01267 | 187 |
| L03294_g_at | | 1 | | Direct Acting | L03294 | 188 |
| L07114_at | | −1 | | Direct Acting | L07114 | 189 |
| L07407_at | | 1 | | Direct Acting | L07407 | 190 |
| L08505_at | | 1 | | Direct Acting | L08505 | 191 |
| L12025_at | | 1 | | Direct Acting | L12025 | 192 |
| L12382_at | | −1 | | Direct Acting | L12382 | 193 |
| L12383_at | | 1 | | Direct Acting | L12383 | 194 |
| L13235UTR#1_f_at | | −1 | | Direct Acting | L13235 | 195 |
| L13600_at | | 1 | | Direct Acting | L13600 | 196 |
| L13635_s_at | | 1 | | Direct Acting | L13635 | 197 |
| L17127_g_at | | 1 | | Direct Acting | L17127 | 198 |
| L19031_at | | −1 | | Direct Acting | L19031 | 199 |
| L19931_at | | 1 | | Direct Acting | L19931 | 200 |
| L19998_at | | −1 | | Direct Acting | L19998 | 201 |
| L20900_at | | 1 | | Direct Acting | L20900 | 202 |
| L22294_at | | −1 | | Direct Acting | L22294 | 203 |
| L22339_at | | −1 | | Direct Acting | L22339 | 204 |
| L22339_g_at | | −1 | | Direct Acting | L22339 | 205 |
| L23148_g_at | | 1 | | Direct Acting | L23148 | 206 |
| L24207_r_at | | −1 | | Direct Acting | L24207 | 207 |
| L27075_g_at | | −1 | | Direct Acting | L27075 | 208 |
| L27843_s_at | | 1 | | Direct Acting | L27843 | 209 |
| L32591mRNA_at | | 1 | | Direct Acting | L32591 | 210 |
| L32591mRNA_g_at | | 1 | | Direct Acting | L32591 | 211 |
| L32601_s_at | | −1 | | Direct Acting | L32601 | 212 |
| L34049_g_at | | −1 | | Direct Acting | L34049 | 213 |
| L38482_g_at | | 1 | | Direct Acting | L38482 | 214 |
| L38615_g_at | | 1 | | Direct Acting | L38615 | 215 |
| L41275cds_s_at | | 1 | | Direct Acting | L41275 | 216 |
| L41685mRNA_at | | 1 | | Direct Acting | L41685 | 217 |
| M11266_at | | −1 | | Direct Acting | M11266 | 218 |
| M11942_s_at | | 1 | | Direct Acting | M11942 | 219 |
| M12156_at | | 1 | | Direct Acting | M12156 | 220 |
| M12919mRNA#2_at | | 1 | | Direct Acting | M12919 | 221 |
| M12919mRNA#2_g_at | | 1 | | Direct Acting | M12919 | 222 |
| M13100cds#3_f_at | | −1 | | Direct Acting | M13100 | 223 |
| M13100cds#4_f_at | | −1 | | Direct Acting | M13100 | 224 |
| M13962mRNA#2_at | | −1 | | Direct Acting | M13962 | 225 |
| M14972_i_at | | 1 | | Direct Acting | M14972 | 226 |
| M15883_g_at | | 1 | | Direct Acting | M15883 | 227 |
| M18363cds_s_at | | −1 | | Direct Acting | M18363 | 228 |
| M21842_at | | −1 | | Direct Acting | M21842 | 229 |
| M22359mRNA_s_at | | −1 | | Direct Acting | M22359 | 230 |
| M22360_s_at | | −1 | | Direct Acting | M22360 | 231 |
| M23601_at | | −1 | | Direct Acting | M23601 | 232 |
| M24067_at | | 1 | | Direct Acting | M24067 | 233 |
| M24604_at | | 1 | | Direct Acting | M24604 | 234 |
| M24604_g_at | | 1 | | Direct Acting | M24604 | 235 |
| M25157mRNA_i_at | | −1 | | Direct Acting | M25157 | 236 |
| M25490_at | | −1 | | Direct Acting | M25490 | 237 |
| M25804_at | | 1 | | Direct Acting | M25804 | 238 |
| M25804_g_at | | 1 | | Direct Acting | M25804 | 239 |
| M27158cds_at | | 1 | | Direct Acting | M27158 | 240 |
| M27207mRNA_s_at | | −1 | | Direct Acting | M27207 | 241 |
| M29249cds_at | | 1 | | Direct Acting | M29249 | 242 |
| M31837_at | | −1 | | Direct Acting | M31837 | 243 |
| M32062_at | | 1 | | Direct Acting | M32062 | 244 |
| M32062_g_at | | 1 | | Direct Acting | M32062 | 245 |
| M33962_at | | 1 | | Direct Acting | M33962 | 246 |
| M36151cds_s_at | | 1 | | Direct Acting | M36151 | 247 |
| M37828_at | | −1 | | Direct Acting | M37828 | 248 |
| M55015cds_s_at | | 1 | | Direct Acting | M55015 | 249 |
| M57728_at | | 1 | | Direct Acting | M57728 | 250 |
| M58041_s_at | | −1 | | Direct Acting | M58041 | 251 |
| M59460mRNA#2_at | | −1 | | Direct Acting | M59460 | 252 |
| M60103_at | | −1 | | Direct Acting | M60103 | 253 |
| M61219_s_at | | 1 | | Direct Acting | M61219 | 254 |
| M63282_at | | 1 | | Direct Acting | M63282 | 255 |
| M64795_f_at | | 1 | | Direct Acting | M64795 | 256 |
| M64862_at | | −1 | | Direct Acting | M64862 | 257 |
| M69246_at | | −1 | | Direct Acting | M69246 | 258 |
| M73808mRNA_at | | 1 | | Direct Acting | M73808 | 259 |
| M75168_at | | 1 | | Direct Acting | M75168 | 260 |
| M76767_s_at | | −1 | | Direct Acting | M76767 | 261 |
| M77245_at | | 1 | | Direct Acting | M77245 | 262 |
| M77479_at | | −1 | | Direct Acting | M77479 | 263 |
| M81183Exon_UTR_g_at | | −1 | | Direct Acting | M81183 | 264 |
| M81855_at | | 1 | | Direct Acting | M81855 | 265 |
| M81920_at | | 1 | | Direct Acting | M81920 | 266 |
| M83675_at | | −1 | | Direct Acting | M83675 | 267 |
| M84719_at | | −1 | | Direct Acting | M84719 | 268 |
| M89945mRNA_at | | −1 | | Direct Acting | M89945 | 269 |
| M89945mRNA_g_at | | −1 | | Direct Acting | M89945 | 270 |
| M91466_at | | −1 | | Direct Acting | M91466 | 271 |
| M91652complete_seq_at | | −1 | | Direct Acting | M91652 | 272 |
| M91652complete_seq_g_at | | −1 | | Direct Acting | M91652 | 273 |
| M93297cds_at | | −1 | | Direct Acting | M93297 | 274 |
| M93401_at | | −1 | | Direct Acting | M93401 | 275 |
| M94043_at | | −1 | | Direct Acting | M94043 | 276 |
| M94555_at | | 1 | | Direct Acting | M94555 | 277 |
| M95591_at | | −1 | | Direct Acting | M95591 | 278 |
| M95591_g_at | | −1 | | Direct Acting | M95591 | 279 |
| M96674_at | | −1 | | Direct Acting | M96674 | 280 |
| rc_AA686164_at | | 1 | | Direct Acting | AA686164 | 281 |
| rc_AA799418_at | | 1 | | Direct Acting | AA799418 | 282 |
| rc_AA799479_at | | 1 | | Direct Acting | AA799479 | 283 |
| rc_AA799481_at | | 1 | | Direct Acting | AA799481 | 284 |
| rc_AA799508_at | | 1 | | Direct Acting | AA799508 | 285 |
| rc_AA799531_at | | 1 | | Direct Acting | AA799531 | 286 |
| rc_AA799531_g_at | | 1 | | Direct Acting | AA799531 | 287 |
| rc_AA799560_at | | −1 | | Direct Acting | AA799560 | 288 |
| rc_AA799672_s_at | | 1 | | Direct Acting | AA799672 | 289 |
| rc_AA799735_at | | 1 | | Direct Acting | AA799735 | 290 |
| rc_AA799788_s_at | | 1 | | Direct Acting | AA799788 | 291 |
| rc_AA799814_at | | 1 | | Direct Acting | AA799814 | 292 |
| rc_AA799893_g_at | | 1 | | Direct Acting | AA799893 | 293 |
| rc_AA799997_at | | −1 | | Direct Acting | AA799997 | 294 |
| rc_AA800017_at | | 1 | | Direct Acting | AA800017 | 295 |
| rc_AA800169_at | | 1 | | Direct Acting | AA800169 | 296 |
| rc_AA800179_at | | 1 | | Direct Acting | AA800179 | 297 |
| rc_AA800218_at | | 1 | | Direct Acting | AA800218 | 298 |
| rc_AA800456_at | | −1 | | Direct Acting | AA800456 | 299 |
| rc_AA800738_at | | 1 | | Direct Acting | AA800738 | 300 |
| rc_AA800739_at | | 1 | | Direct Acting | AA800739 | 301 |
| rc_AA800750_f_at | | −1 | | Direct Acting | AA800750 | 302 |
| rc_AA800753_at | | 1 | | Direct Acting | AA800753 | 303 |
| rc_AA800797_at | | −1 | | Direct Acting | AA800797 | 304 |
| rc_AA800912_g_at | | 1 | | Direct Acting | AA800912 | 305 |
| rc_AA817846_at | | −1 | | Direct Acting | AA817846 | 306 |
| rc_AA817854_s_at | | −1 | | Direct Acting | AA817854 | 307 |
| rc_AA817987_f_at | | −1 | | Direct Acting | AA817987 | 308 |
| rc_AA818072_s_at | | 1 | | Direct Acting | AA818072 | 309 |
| rc_AA818122_f_at | | −1 | | Direct Acting | AA818122 | 310 |
| rc_AA818951_at | | 1 | | Direct Acting | AA818951 | 311 |
| rc_AA819776_f_at | | 1 | | Direct Acting | AA819776 | 312 |
| rc_AA849722_at | | 1 | | Direct Acting | AA849722 | 313 |
| rc_AA852004_s_at | | −1 | | Direct Acting | AA852004 | 314 |
| rc_AA858879_at | | 1 | | Direct Acting | AA858879 | 315 |
| rc_AA859648_at | | 1 | | Direct Acting | AA859648 | 316 |
| rc_AA859652_at | | 1 | | Direct Acting | AA859652 | 317 |
| rc_AA859663_at | | −1 | | Direct Acting | AA859663 | 318 |
| rc_AA859680_at | | 1 | | Direct Acting | AA859680 | 319 |
| rc_AA859680_g_at | | 1 | | Direct Acting | AA859680 | 320 |
| rc_AA859722_at | | 1 | | Direct Acting | AA859722 | 321 |
| rc_AA859980_at | | −1 | | Direct Acting | AA859980 | 322 |
| rc_AA859980_g_at | | −1 | | Direct Acting | AA859980 | 323 |
| rc_AA860030_s_at | | 1 | | Direct Acting | AA860030 | 324 |
| rc_AA866264_s_at | | −1 | | Direct Acting | AA866264 | 325 |
| rc_AA866426_at | | −1 | | Direct Acting | AA866426 | 326 |
| rc_AA874791_at | | 1 | | Direct Acting | AA874791 | 327 |
| rc_AA874802_s_at | | −1 | | Direct Acting | AA874802 | 328 |
| rc_AA874889_g_at | | 1 | | Direct Acting | AA874889 | 329 |
| rc_AA875054_at | | 1 | | Direct Acting | AA875054 | 330 |
| rc_AA875126_g_at | | 1 | | Direct Acting | AA875126 | 331 |
| rc_AA875205_at | | 1 | | Direct Acting | AA875205 | 332 |
| rc_AA875205_g_at | | 1 | | Direct Acting | AA875205 | 333 |
| rc_AA875511_at | | −1 | | Direct Acting | AA875511 | 334 |
| rc_AA875531_s_at | | −1 | | Direct Acting | AA875531 | 335 |
| rc_AA875537_at | | 1 | | Direct Acting | AA875537 | 336 |
| rc_AA875563_at | | 1 | | Direct Acting | AA875563 | 337 |
| rc_AA875620_g_at | | 1 | | Direct Acting | AA875620 | 338 |
| rc_AA891226_s_at | | 1 | | Direct Acting | AA891226 | 339 |
| rc_AA891553_at | | 1 | | Direct Acting | AA891553 | 340 |
| rc_AA891689_at | | 1 | | Direct Acting | AA891689 | 341 |
| rc_AA891689_g_at | | 1 | | Direct Acting | AA891689 | 342 |
| rc_AA891739_at | | −1 | | Direct Acting | AA891739 | 343 |
| rc_AA891785_at | | 1 | | Direct Acting | AA891785 | 344 |
| rc_AA891790_at | | 1 | | Direct Acting | AA891790 | 345 |
| rc_AA891829_at | | 1 | | Direct Acting | AA891829 | 346 |
| rc_AA891838_at | | 1 | | Direct Acting | AA891838 | 347 |
| rc_AA891998_i_at | | 1 | | Direct Acting | AA891998 | 348 |
| rc_AA892006_at | | 1 | | Direct Acting | AA892006 | 349 |
| rc_AA892010_g_at | | 1 | | Direct Acting | AA892010 | 350 |
| rc_AA892014_r_at | | 1 | | Direct Acting | AA892014 | 351 |
| rc_AA892027_at | | −1 | | Direct Acting | AA892027 | 352 |
| rc_AA892053_at | | 1 | | Direct Acting | AA892053 | 353 |
| rc_AA892120_at | | 1 | | Direct Acting | AA892120 | 354 |
| rc_AA892154_g_at | | −1 | | Direct Acting | AA892154 | 355 |
| rc_AA892248_g_at | | −1 | | Direct Acting | AA892248 | 356 |
| rc_AA892251_at | | −1 | | Direct Acting | AA892251 | 357 |
| rc_AA892333_at | | 1 | | Direct Acting | AA892333 | 358 |
| rc_AA892367_i_at | | 1 | | Direct Acting | AA892367 | 359 |
| rc_AA892378_at | | 1 | | Direct Acting | AA892378 | 360 |
| rc_AA892500_at | | −1 | | Direct Acting | AA892500 | 361 |
| rc_AA892562_at | | 1 | | Direct Acting | AA892562 | 362 |
| rc_AA892562_g_at | | 1 | | Direct Acting | AA892562 | 363 |
| rc_AA892582_s_at | | 1 | | Direct Acting | AA892582 | 364 |
| rc_AA892598_at | | 1 | | Direct Acting | AA892598 | 365 |
| rc_AA892598_g_at | | 1 | | Direct Acting | AA892598 | 366 |
| rc_AA892602_at | | 1 | | Direct Acting | AA892602 | 367 |
| rc_AA892680_at | | 1 | | Direct Acting | AA892680 | 368 |
| rc_AA892799_i_at | | 1 | | Direct Acting | AA892799 | 369 |
| rc_AA892799_r_at | | −1 | | Direct Acting | AA892799 | 370 |
| rc_AA892828_at | | −1 | | Direct Acting | AA892828 | 371 |
| rc_AA892828_g_at | | −1 | | Direct Acting | AA892828 | 372 |
| rc_AA892832_at | | −1 | | Direct Acting | AA892832 | 373 |
| rc_AA892855_at | | −1 | | Direct Acting | AA892855 | 374 |
| rc_AA892861_at | | −1 | | Direct Acting | AA892861 | 375 |
| rc_AA892950_at | | 1 | | Direct Acting | AA892950 | 376 |
| rc_AA892986_at | | −1 | | Direct Acting | AA892986 | 377 |
| rc_AA893032_at | | −1 | | Direct Acting | AA893032 | 378 |
| rc_AA893199_at | | 1 | | Direct Acting | AA893199 | 379 |
| rc_AA893235_at | | 1 | | Direct Acting | AA893235 | 380 |
| rc_AA893239_at | | −1 | | Direct Acting | AA893239 | 381 |
| rc_AA893242_g_at | | −1 | | Direct Acting | AA893242 | 382 |
| rc_AA893280_at | | 1 | | Direct Acting | AA893280 | 383 |
| rc_AA893325_at | | −1 | | Direct Acting | AA893325 | 384 |
| rc_AA893366_at | | −1 | | Direct Acting | AA893366 | 385 |
| rc_AA893384_g_at | | −1 | | Direct Acting | AA893384 | 386 |
| rc_AA893471_s_at | | −1 | | Direct Acting | AA893471 | 387 |
| rc_AA893495_at | | −1 | | Direct Acting | AA893495 | 388 |
| rc_AA893517_at | | 1 | | Direct Acting | AA893517 | 389 |
| rc_AA893532_at | | 1 | | Direct Acting | AA893532 | 390 |
| rc_AA893562_at | | 1 | | Direct Acting | AA893562 | 391 |
| rc_AA893584_at | | 1 | | Direct Acting | AA893584 | 392 |
| rc_AA893690_at | | 1 | | Direct Acting | AA893690 | 393 |
| rc_AA893770_g_at | | 1 | | Direct Acting | AA893770 | 394 |
| rc_AA894027_at | | −1 | | Direct Acting | AA894027 | 395 |
| rc_AA894086_g_at | | 1 | | Direct Acting | AA894086 | 396 |
| rc_AA894258_at | | −1 | | Direct Acting | AA894258 | 397 |
| rc_AA894298_s_at | | 1 | | Direct Acting | AA894298 | 398 |
| rc_AA900476_at | | −1 | | Direct Acting | AA900476 | 399 |
| rc_AA924267_s_at | | 1 | | Direct Acting | AA924267 | 400 |
| rc_AA924289_s_at | | −1 | | Direct Acting | AA924289 | 401 |
| rc_AA924326_s_at | | 1 | | Direct Acting | AA924326 | 402 |
| rc_AA926193_at | | −1 | | Direct Acting | AA926193 | 403 |
| rc_AA944156_s_at | | 1 | | Direct Acting | AA944156 | 404 |
| rc_AA944397_at | | 1 | | Direct Acting | AA944397 | 405 |
| rc_AA945082_at | | 1 | | Direct Acting | AA945082 | 406 |
| rc_AA945867_at | | 1 | | Direct Acting | AA945867 | 407 |
| rc_AA946532_at | | −1 | | Direct Acting | AA946532 | 408 |
| rc_AA956958_at | | 1 | | Direct Acting | AA956958 | 409 |
| rc_AA963449_s_at | | −1 | | Direct Acting | AA963449 | 410 |
| rc_AA963839_s_at | | −1 | | Direct Acting | AA963839 | 411 |
| rc_AA965147_at | | 1 | | Direct Acting | AA965147 | 412 |
| rc_AA997614_s_at | | −1 | | Direct Acting | AA997614 | 413 |
| rc_AI008074_s_at | | 1 | | Direct Acting | AI008074 | 414 |
| rc_AI008131_s_at | | 1 | | Direct Acting | AI008131 | 415 |
| rc_AI009338_at | | −1 | | Direct Acting | AI009338 | 416 |
| rc_AI009806_at | | 1 | | Direct Acting | AI009806 | 417 |
| rc_AI011998_at | | 1 | | Direct Acting | AI011998 | 418 |
| rc_AI012595_at | | 1 | | Direct Acting | AI012595 | 419 |
| rc_AI012604_at | | 1 | | Direct Acting | AI012604 | 420 |
| rc_AI013513_at | | 1 | | Direct Acting | AI013513 | 421 |
| rc_AI014091_at | | −1 | | Direct Acting | AI014091 | 422 |
| rc_AI014163_at | | 1 | | Direct Acting | AI014163 | 423 |
| rc_AI031019_g_at | | 1 | | Direct Acting | AI031019 | 424 |
| rc_AI044900_s_at | | −1 | | Direct Acting | AI044900 | 425 |
| rc_AI044985_g_at | | −1 | | Direct Acting | AI044985 | 426 |
| rc_AI045395_at | | −1 | | Direct Acting | AI045395 | 427 |
| rc_AI070295_at | | 1 | | Direct Acting | AI070295 | 428 |
| rc_AI070295_g_at | | 1 | | Direct Acting | AI070295 | 429 |
| rc_AI102103_g_at | | 1 | | Direct Acting | AI102103 | 430 |
| rc_AI105348_f_at | | 1 | | Direct Acting | AI105348 | 431 |
| rc_AI105348_i_at | | 1 | | Direct Acting | AI105348 | 432 |
| rc_AI111401_s_at | | 1 | | Direct Acting | AI111401 | 433 |
| rc_AI137790_at | | 1 | | Direct Acting | AI137790 | 434 |
| rc_AI169695_f_at | | −1 | | Direct Acting | AI169695 | 435 |
| rc_AI169735_g_at | | −1 | | Direct Acting | AI169735 | 436 |
| rc_AI170608_at | | 1 | | Direct Acting | AI170608 | 437 |
| rc_AI171966_at | | 1 | | Direct Acting | AI171966 | 438 |
| rc_AI172476_at | | 1 | | Direct Acting | AI172476 | 439 |
| rc_AI175486_at | | 1 | | Direct Acting | AI175486 | 440 |
| rc_AI175959_at | | 1 | | Direct Acting | AI175959 | 441 |
| rc_AI176488_at | | −1 | | Direct Acting | AI176488 | 442 |
| rc_AI176595_s_at | | 1 | | Direct Acting | AI176595 | 443 |
| rc_AI177161_at | | −1 | | Direct Acting | AI177161 | 444 |
| rc_AI177161_g_at | | −1 | | Direct Acting | AI177161 | 445 |
| rc_AI177986_at | | 1 | | Direct Acting | AI177986 | 446 |
| rc_AI178135_at | | 1 | | Direct Acting | AI178135 | 447 |
| rc_AI178828_i_at | | 1 | | Direct Acting | AI178828 | 448 |
| rc_AI179610_at | | 1 | | Direct Acting | AI179610 | 449 |
| rc_AI180442_at | | −1 | | Direct Acting | AI180442 | 450 |
| rc_AI228738_s_at | | 1 | | Direct Acting | AI228738 | 451 |
| rc_AI229637_at | | 1 | | Direct Acting | AI229637 | 452 |
| rc_AI230260_s_at | | 1 | | Direct Acting | AI230260 | 453 |
| rc_AI230294_at | | −1 | | Direct Acting | AI230294 | 454 |
| rc_AI230614_s_at | | 1 | | Direct Acting | AI230614 | 455 |
| rc_AI230712_at | | 1 | | Direct Acting | AI230712 | 456 |
| rc_AI231007_at | | 1 | | Direct Acting | AI231007 | 457 |
| rc_AI231807_g_at | | 1 | | Direct Acting | AI231807 | 458 |
| rc_AI232783_s_at | | −1 | | Direct Acting | AI232783 | 459 |
| rc_AI234604_s_at | | 1 | | Direct Acting | AI234604 | 460 |
| rc_AI235631_at | | 1 | | Direct Acting | AI235631 | 461 |
| rc_AI235890_s_at | | −1 | | Direct Acting | AI235890 | 462 |
| rc_AI236597_at | | 1 | | Direct Acting | AI236597 | 463 |
| rc_AI236601_at | | 1 | | Direct Acting | AI236601 | 464 |
| rc_AI237535_s_at | | 1 | | Direct Acting | AI237535 | 465 |
| rc_AI638948_at | | −1 | | Direct Acting | AI638948 | 466 |
| rc_AI638966_r_at | | −1 | | Direct Acting | AI638966 | 467 |
| rc_AI639008_at | | 1 | | Direct Acting | AI639008 | 468 |
| rc_AI639029_s_at | | 1 | | Direct Acting | AI639029 | 469 |
| rc_AI639067_at | | −1 | | Direct Acting | AI639067 | 470 |
| rc_AI639167_at | | 1 | | Direct Acting | AI639167 | 471 |
| rc_AI639185_s_at | | −1 | | Direct Acting | AI639185 | 472 |
| rc_AI639393_at | | 1 | | Direct Acting | AI639393 | 473 |
| rc_AI639488_at | | 1 | | Direct Acting | AI639488 | 474 |
| rc_AI639518_g_at | | 1 | | Direct Acting | AI639518 | 475 |
| rc_H31287_g_at | | 1 | | Direct Acting | H31287 | 476 |
| rc_H31351_at | | 1 | | Direct Acting | H31351 | 477 |
| rc_H31722_at | | 1 | | Direct Acting | H31722 | 478 |
| rc_H31976_at | | 1 | | Direct Acting | H31976 | 479 |
| rc_H31982_at | | 1 | | Direct Acting | H31982 | 480 |
| rc_H33426_at | | −1 | | Direct Acting | H33426 | 481 |
| rc_H33426_g_at | | −1 | | Direct Acting | H33426 | 482 |
| rc_H33491_at | | −1 | | Direct Acting | H33491 | 483 |
| S46785_at | | −1 | | Direct Acting | S46785 | 484 |
| S46785_g_at | | −1 | | Direct Acting | S46785 | 485 |
| S55224_s_at | | 1 | | Direct Acting | S55224 | 486 |
| S61868_g_at | | 1 | | Direct Acting | S61868 | 487 |
| S66024_at | | 1 | | Direct Acting | S66024 | 488 |
| S69874_s_at | | 1 | | Direct Acting | S69874 | 489 |
| S71021_s_at | | 1 | | Direct Acting | S71021 | 490 |
| S72506_s_at | | 1 | | Direct Acting | S72506 | 491 |
| S76054_s_at | | 1 | | Direct Acting | S76054 | 492 |
| S76489_s_at | | −1 | | Direct Acting | S76489 | 493 |
| S79213_at | | 1 | | Direct Acting | S79213 | 494 |
| S79820_at | | 1 | | Direct Acting | S79820 | 495 |
| S80456_s_at | | 1 | | Direct Acting | S80456 | 496 |
| S82820mRNA_s_at | | 1 | | Direct Acting | S82820 | 497 |
| S85184_at | | 1 | | Direct Acting | S85184 | 498 |
| S85184_g_at | | 1 | | Direct Acting | S85184 | 499 |
| U01146_s_at | | 1 | | Direct Acting | U01146 | 500 |
| U01344_at | | −1 | | Direct Acting | U01344 | 501 |
| U03390_at | | 1 | | Direct Acting | U03390 | 502 |
| U05014_g_at | | 1 | | Direct Acting | U05014 | 503 |
| U05784_s_at | | 1 | | Direct Acting | U05784 | 504 |
| U07201_at | | 1 | | Direct Acting | U07201 | 505 |
| U08141_at | | −1 | | Direct Acting | U08141 | 506 |
| U12268_at | | −1 | | Direct Acting | U12268 | 507 |
| U14746_at | | 1 | | Direct Acting | U14746 | 508 |
| U17035_s_at | | 1 | | Direct Acting | U17035 | 509 |
| U17697_s_at | | −1 | | Direct Acting | U17697 | 510 |
| U18729_at | | 1 | | Direct Acting | U18729 | 511 |
| U21101_at | | −1 | | Direct Acting | U21101 | 512 |
| U21719mRNA_s_at | | 1 | | Direct Acting | U21719 | 513 |
| U21871_at | | 1 | | Direct Acting | U21871 | 514 |
| U24174_at | | 1 | | Direct Acting | U24174 | 515 |
| U28504_at | | −1 | | Direct Acting | U28504 | 516 |
| U29873_at | | −1 | | Direct Acting | U29873 | 517 |
| U30186_at | | 1 | | Direct Acting | U30186 | 518 |
| U31777_g_at | | 1 | | Direct Acting | U31777 | 519 |
| U31866_at | | −1 | | Direct Acting | U31866 | 520 |
| U33500_g_at | | 1 | | Direct Acting | U33500 | 521 |
| U33541cds_at | | −1 | | Direct Acting | U33541 | 522 |
| U36482_g_at | | −1 | | Direct Acting | U36482 | 523 |
| U38253_at | | 1 | | Direct Acting | U38253 | 524 |
| U38253_g_at | | 1 | | Direct Acting | U38253 | 525 |
| U40004_s_at | | −1 | | Direct Acting | U40004 | 526 |
| U44948_at | | 1 | | Direct Acting | U44948 | 527 |
| U50412_at | | −1 | | Direct Acting | U50412 | 528 |
| U52530_s_at | | −1 | | Direct Acting | U52530 | 529 |
| U53873cds_at | | −1 | | Direct Acting | U53873 | 530 |
| U55815_at | | 1 | | Direct Acting | U55815 | 531 |
| U60416_at | | 1 | | Direct Acting | U60416 | 532 |
| U60882_at | | 1 | | Direct Acting | U60882 | 533 |
| U63923_at | | 1 | | Direct Acting | U63923 | 534 |
| U64705cds_f_at | | 1 | | Direct Acting | U64705 | 535 |
| U66322_at | | −1 | | Direct Acting | U66322 | 536 |
| U67915_at | | −1 | | Direct Acting | U67915 | 537 |
| U68168_at | | −1 | | Direct Acting | U68168 | 538 |
| U72349_at | | 1 | | Direct Acting | U72349 | 539 |
| U73174_at | | 1 | | Direct Acting | U73174 | 540 |
| U75210_s_at | | −1 | | Direct Acting | U75210 | 541 |
| U75405UTR#1_f_at | | −1 | | Direct Acting | U75405 | 542 |
| U75917_at | | 1 | | Direct Acting | U75917 | 543 |
| U76714_at | | 1 | | Direct Acting | U76714 | 544 |
| U77918_at | | 1 | | Direct Acting | U77918 | 545 |
| U83896_at | | 1 | | Direct Acting | U83896 | 546 |
| U84410_s_at | | −1 | | Direct Acting | U84410 | 547 |
| U88036_at | | −1 | | Direct Acting | U88036 | 548 |
| U91561_g_at | | 1 | | Direct Acting | U91561 | 549 |
| U96490_at | | 1 | | Direct Acting | U96490 | 550 |
| V01225mRNA_s_at | | −1 | | Direct Acting | V01225 | 551 |
| V01274_at | | −1 | | Direct Acting | V01274 | 552 |
| X02610_at | | 1 | | Direct Acting | X02610 | 553 |
| X02741_s_at | | 1 | | Direct Acting | X02741 | 554 |
| X04069_at | | −1 | | Direct Acting | X04069 | 555 |
| X04267_at | | 1 | | Direct Acting | X04267 | 556 |
| X05137_at | | −1 | | Direct Acting | X05137 | 557 |
| X05472cds#1_s_at | | −1 | | Direct Acting | X05472 | 558 |
| X06423_g_at | | 1 | | Direct Acting | X06423 | 559 |
| X06801cds_f_at | | 1 | | Direct Acting | X06801 | 560 |
| X07259cds_s_at | | 1 | | Direct Acting | X07259 | 561 |
| X07551cds_s_at | | 1 | | Direct Acting | X07551 | 562 |
| X07686cds_s_at | | −1 | | Direct Acting | X07686 | 563 |
| X07944exon#1- | | 1 | | Direct Acting | X07944 | 564 |
| 12_s_at | | | | | | |
| X08056cds_s_at | | −1 | | Direct Acting | X08056 | 565 |
| X12367cds_s_at | | −1 | | Direct Acting | X12367 | 566 |
| X13044_at | | 1 | | Direct Acting | X13044 | 567 |
| X13058_at | | 1 | | Direct Acting | X13058 | 568 |
| X13527cds_s_at | | −1 | | Direct Acting | X13527 | 569 |
| X14181cds_s_at | | 1 | | Direct Acting | X14181 | 570 |
| X14254cds_g_at | | 1 | | Direct Acting | X14254 | 571 |
| X15580complete_seq_s_at | | −1 | | Direct Acting | X15580 | 572 |
| X16038exon_s_at | | 1 | | Direct Acting | X16038 | 573 |
| X16043cds_at | | 1 | | Direct Acting | X16043 | 574 |
| X16044cds_s_at | | 1 | | Direct Acting | X16044 | 575 |
| X16554_at | | 1 | | Direct Acting | X16554 | 576 |
| X17053mRNA_s_at | | 1 | | Direct Acting | X17053 | 577 |
| X52619_at | | 1 | | Direct Acting | X52619 | 578 |
| X52815cds_f_at | | 1 | | Direct Acting | X52815 | 579 |
| X53581cds#3_f_at | | −1 | | Direct Acting | X53581 | 580 |
| X53588_at | | −1 | | Direct Acting | X53588 | 581 |
| X55286_at | | 1 | | Direct Acting | X55286 | 582 |
| X57432cds_s_at | | 1 | | Direct Acting | X57432 | 583 |
| X57523_at | | 1 | | Direct Acting | X57523 | 584 |
| X57523_g_at | | 1 | | Direct Acting | X57523 | 585 |
| X58465mRNA_at | | 1 | | Direct Acting | X58465 | 586 |
| X58465mRNA_g_at | | 1 | | Direct Acting | X58465 | 587 |
| X59859_i_at | | 1 | | Direct Acting | X59859 | 588 |
| X60212_i_at | | 1 | | Direct Acting | X60212 | 589 |
| X60769mRNA_at | | 1 | | Direct Acting | X60769 | 590 |
| X61296cds#2_f_at | | −1 | | Direct Acting | X61296 | 591 |
| X62086mRNA_s_at | | −1 | | Direct Acting | X62086 | 592 |
| X62145cds_at | | 1 | | Direct Acting | X62145 | 593 |
| X62295cds_s_at | | −1 | | Direct Acting | X62295 | 594 |
| X62875mRNA_g_at | | 1 | | Direct Acting | X62875 | 595 |
| X64052cds_f_at | | −1 | | Direct Acting | X64052 | 596 |
| X66870_at | | 1 | | Direct Acting | X66870 | 597 |
| X67788_at | | 1 | | Direct Acting | X67788 | 598 |
| X69903_at | | −1 | | Direct Acting | X69903 | 599 |
| X70369_s_at | | −1 | | Direct Acting | X70369 | 600 |
| X70871_at | | 1 | | Direct Acting | X70871 | 601 |
| X74565cds_g_at | | 1 | | Direct Acting | X74565 | 602 |
| X76453_at | | −1 | | Direct Acting | X76453 | 603 |
| X77235_at | | 1 | | Direct Acting | X77235 | 604 |
| X77932_at | | −1 | | Direct Acting | X77932 | 605 |
| X77934cds_at | | −1 | | Direct Acting | X77934 | 606 |
| X78327_at | | 1 | | Direct Acting | X78327 | 607 |
| X78997_at | | 1 | | Direct Acting | X78997 | 608 |
| X79081mRNA_f_at | | −1 | | Direct Acting | X79081 | 609 |
| X81448cds_at | | 1 | | Direct Acting | X81448 | 610 |
| X84210complete_seq_s_at | | −1 | | Direct Acting | X84210 | 611 |
| X89225cds_s_at | | 1 | | Direct Acting | X89225 | 612 |
| X95189_at | | −1 | | Direct Acting | X95189 | 613 |
| X95986mRNA#1_f_at | | 1 | | Direct Acting | X95986 | 614 |
| X97772_at | | 1 | | Direct Acting | X97772 | 615 |
| X97772_g_at | | 1 | | Direct Acting | X97772 | 616 |
| Y00396mRNA_at | | 1 | | Direct Acting | Y00396 | 617 |
| Y00396mRNA_g_at | | 1 | | Direct Acting | Y00396 | 618 |
| Y08355cds#2_at | | 1 | | Direct Acting | Y08355 | 619 |
| Y09333_at | | 1 | | Direct Acting | Y09333 | 620 |
| Y09365cds_s_at | | 1 | | Direct Acting | Y09365 | 621 |
| Y12635_at | | 1 | | Direct Acting | Y12635 | 622 |
| Y14933mRNA_s_at | | 1 | | Direct Acting | Y14933 | 623 |
| Y17295cds_s_at | | 1 | | Direct Acting | Y17295 | 624 |
| Z36944cds_at | | 1 | | Direct Acting | Z36944 | 625 |
| Z83757mRNA_at | | −1 | | Direct Acting | Z83757 | 626 |
| Z83757mRNA_g_at | | −1 | | Direct Acting | Z83757 | 627 |
| J03863_at | 1 | | 1 | Cholestatic/ | J03863 | 628 |
| | | | | Steatotic | | |
| J05460_s_at | 1 | | −1 | Cholestatic/ | J05460 | 629 |
| | | | | Steatotic | | |
| X13119cds_s_at | 1 | | 1 | Cholestatic/ | X13119 | 630 |
| | | | | Steatotic | | |
| AF020618_g_at | 1 | 1 | | Cholestatic/ | AF020618 | 631 |
| | | | | Direct Acting | | |
| AF039832_at | 1 | 1 | | Cholestatic/ | AF039832 | 632 |
| | | | | Direct Acting | | |
| AF086624_s_at | 1 | 1 | | Cholestatic/ | AF086624 | 633 |
| | | | | Direct Acting | | |
| AF089825_at | −1 | −1 | | Cholestatic/ | AF089825 | 634 |
| | | | | Direct Acting | | |
| D12769_g_at | 1 | 1 | | Cholestatic/ | D12769 | 635 |
| | | | | Direct Acting | | |
| D37920_at | −1 | −1 | | Cholestatic/ | D37920 | 636 |
| | | | | Direct Acting | | |
| D86580_at | 1 | −1 | | Cholestatic/ | D86580 | 637 |
| | | | | Direct Acting | | |
| J02722cds_at | 1 | 1 | | Cholestatic/ | J02722 | 638 |
| | | | | Direct Acting | | |
| J04171_at | 1 | 1 | | Cholestatic/ | J04171 | 639 |
| | | | | Direct Acting | | |
| K03041mRNA_s_at | 1 | −1 | | Cholestatic/ | K03041 | 640 |
| | | | | Direct Acting | | |
| L37333_s_at | 1 | −1 | | Cholestatic/ | L37333 | 641 |
| | | | | Direct Acting | | |
| M57507_at | −1 | −1 | | Cholestatic/ | M57507 | 642 |
| | | | | Direct Acting | | |
| M60921_at | 1 | 1 | | Cholestatic/ | M60921 | 643 |
| | | | | Direct Acting | | |
| M96548_at | 1 | 1 | | Cholestatic/ | M96548 | 644 |
| | | | | Direct Acting | | |
| rc_AA799861_g_at | −1 | 1 | | Cholestatic/ | AA799861 | 645 |
| | | | | Direct Acting | | |
| rc_AA800678_g_at | −1 | −1 | | Cholestatic/ | AA800678 | 646 |
| | | | | Direct Acting | | |
| rc_AA891944_at | −1 | −1 | | Cholestatic/ | AA891944 | 647 |
| | | | | Direct Acting | | |
| rc_AA900505_at | 1 | 1 | | Cholestatic/ | AA900505 | 648 |
| | | | | Direct Acting | | |
| rc_AI009098_at | −1 | 1 | | Cholestatic/ | AI009098 | 649 |
| | | | | Direct Acting | | |
| rc_AI112173_at | 1 | 1 | | Cholestatic/ | AI112173 | 650 |
| | | | | Direct Acting | | |
| rc_H31707_at | −1 | 1 | | Cholestatic/ | H31707 | 651 |
| | | | | Direct Acting | | |
| S61868_at | 1 | 1 | | Cholestatic/ | S61868 | 652 |
| | | | | Direct Acting | | |
| U14005exon#1_s_at | −1 | −1 | | Cholestatic/ | U14005 | 653 |
| | | | | Direct Acting | | |
| U42627_at | 1 | −1 | | Cholestatic/ | U42627 | 654 |
| | | | | Direct Acting | | |
| X07266cds_s_at | 1 | 1 | | Cholestatic/ | X07266 | 655 |
| | | | | Direct Acting | | |
| X63594cds_at | 1 | 1 | | Cholestatic/ | X63594 | 656 |
| | | | | Direct Acting | | |
| X96437mRNA_g_at | 1 | 1 | | Cholestatic/ | X96437 | 657 |
| | | | | Direct Acting | | |
| AF000942_at | −1 | | | Cholestatic | AF000942 | 658 |
| AF075382_at | 1 | | | Cholestatic | AF075382 | 659 |
| D00403_g_at | 1 | | | Cholestatic | D00403 | 660 |
| J03865mRNA_f_at | 1 | | | Cholestatic | J03865 | 661 |
| K03243mRNA_s_at | 1 | | | Cholestatic | K03243 | 662 |
| L13619_at | 1 | | | Cholestatic | L13619 | 663 |
| L13619_g_at | 1 | | | Cholestatic | L13619 | 664 |
| M11794cds#2_f_at | −1 | 1 | 1 | Cholestatic | M11794 | 665 |
| M33962_g_at | 1 | | | Cholestatic | M33962 | 666 |
| M63122_at | −1 | 1 | 1 | Cholestatic | M63122 | 667 |
| rc_AA685221_at | −1 | | | Cholestatic | AA685221 | 668 |
| rc_AA800613_at | 1 | | | Cholestatic | AA800613 | 669 |
| rc_AA866383_at | 1 | | | Cholestatic | AA866383 | 670 |
| rc_AA893192_at | 1 | | | Cholestatic | AA893192 | 671 |
| rc_AA893602_at | −1 | | | Cholestatic | AA893602 | 672 |
| rc_AA946108_at | 1 | −1 | −1 | Cholestatic | AA946108 | 673 |
| rc_AI102562_at | −1 | 1 | 1 | Cholestatic | AI102562 | 674 |
| rc_AI176456_at | −1 | 1 | 1 | Cholestatic | AI176456 | 675 |
| rc_AI176662_s_at | 1 | | | Cholestatic | AI176662 | 676 |
| rc_AI639141_at | 1 | | | Cholestatic | AI639141 | 677 |
| rc_H31118_at | 1 | | | Cholestatic | H31118 | 678 |
| U15211_g_at | −1 | | | Cholestatic | U15211 | 679 |
| X63594cds_g_at | 1 | | | Cholestatic | X63594 | 680 |

TABLE 4
Candidate Marker
Genes

| # | Name | Affymetrix IDs | Acc. Numbers | Comment | SEQ ID NO |
|---|---|---|---|---|---|
| 1 | PEG-3 | AF020618_at; | AF020618 | Early cell stress | 84; |
| | | AF020618_g_at | | marker | 631 |
| 2 | GADD45 | L32591mRNA_at; | L32591; | Stress marker | 210; |
| | | L32591mRNA_g_at; | RNGADD45X | | 211 |
| | | rc_AI070295_at; | | | |
| | | rc_AI070295_g_at | | | |
| 3 | GADD153 | U30186_at | U30186 | Stress marker | 518 |
| 4 | PC3 (BTG2) | M60921_at; | M60921; | Stress marker | 643 |
| | | M60921_g_at; | | | 6 |
| | | rc_AA944156_s_at | AA944156 | | 404 |
| 5 | PC4 (IFR1) | rc_AI014163_at | AI014163 | Stress marker | 423 |
| 6 | CYP2b2 | M13234cds_f_at; | M13234; | Induced by | 741 |
| | | J00728cds_f_at | J00728 | some steatotic | |
| | | | | compounds | |
| 7 | AH- | AF082125_s_at; | AF082125; | Induced by | 109 |
| | Receptor | AF082124_s_at | AF082124 | some steatotic | |
| | | | | compounds | |
| 8 | IGFBP-1 | M58634_at | M58634 | Stress marker | 5 |
| 9 | TRAP | Z14030_at | Z14030 | Induced by | 860 |
| | | | | some direct | |
| | | | | acting | |
| | | | | compounds | |
| 10 | GAPDH | M17701_s_at | P04797 | House-keeping | |
| | | | | gene | |
| 11 | Amyloid_A4 | X07648cds_at | X07648 | Induced by | 66 |
| | | | | some steatotic | |
| | | | | compounds | |
| 12 | Glutathione | U73174_g_at | U73174 | | 540 |
| | reductase | | | | |
| 13 | Carboxyl | AB010635_s_at | AB010635 | Induced by | 40 |
| | esterase | | | some steatotic | |
| | | | | compounds | |
| 14 | CYP3A1 | D13912_s_at | D13912 | Induced by | 861 |
| | | | | some steatotic | |
| | | | | compounds | |
| 15 | CYP9B | L00320cds_f_at | L00320 | Induced by | 793 |
| | | | | some steatotic | |
| | | | | compounds | |
| 16 | UDP- | M13506_at | RNUD2A10; | Induced by | 862 |
| | glucuronosyltransferase | | M35086; | some steatotic | |
| | 2B | | J05482 | compounds | |
| 17 | EGR1 | AF023087 | AF023087; | | 1 |
| | (Krox24) | | M18416; | | |
| | | | U7539; | | |
| | | | U75398; | | |
| | | | AI176662; | | |
| | | | RNNGFIA | | |

TABLE 5
PCR
Validation

| | | | | PC3 | | | |
|---|---|---|---|---|---|---|---|
| Treatment | Toxic | AH-R | | (BTG2) | | CYP2B2 | |
| Group | manifestation | RT-PCR | Affymetrix | RT-PCR | Affymetrix | RT-PCR | Affymetrix |
| Control, 6 H | Control | 1.0 | 1.0 | 1.0 | 1.0 | 1.0 | 1.0 |
| Ro65-7199, | Steatotic | 2.7 | 8.0 | 0.6 | 0.1 | 31.2 | 3.2 |
| 6H | | | | | | | |
| Ro66-0074, | Non-toxic | 1.7 | 1.0 | 0.5 | 0.4 | 8.4 | −1.2 |
| 6H | | | | | | | |
| Control, 24 H | Control | 1.0 | 1.0 | 1.0 | 1.0 | 1.0 | 1.0 |
| Ro65-7199, | Steatotic | 1.1 | 4.5 | 2.7 | 5.7 | 15.9 | 3.4 |
| 24 H | | | | | | | |
| Ro66-0074, | Non-toxic | 1.2 | 1.0 | 0.7 | 0.6 | 1.3 | 1.1 |
| 24 H | | | | | | | |
| Control, 7 D | Control | 1.0 | 1.0 | 1.0 | 1.0 | 1.0 | 1.0 |
| Ro65-7199, | Steatotic | 2.0 | 2.8 | 0.0 | 0.7 | 3.9 | 4.1 |
| 7 D | | | | | | | |

Ro66-0074: 4-(2-Bromo-6-pyrrolidin-1-yl-pyridine-4-sulfonyl)-phenylamine
Ro65-7199: (4-Amino-N-(6-bromo-1 H-indol-4-yl)-benzenesulfonamide.

TABLE 6

| Gene | Acc. | | SEQ ID | | SEQ ID |
|---|---|---|---|---|---|
| Name | Number | Forward Primer | NO | Reverse Primer | NO |
| PEG-3 | AF020618 | GCGGCTCAGATCTTTC | 830 | AGTGGTCACATCT | 831 |
| | | AAAGC | | TGGCTGAGG | |
| GADD45 | L32591; | ATAACTGTCGGCGTGT | 832 | ATCCATGTAGCGA | 833 |
| | RNGADD | ACGAGG | | CTTTCCCG | |
| | 45X | | | | |
| GADD153 | U30186 | TTTCGCCTTTGAGACA | 834 | TGACCACTCTGTT | 835 |
| | | GTGTCC | | TCCGTTTCC | |
| PC3 | M60921; | TTGGCCTAGCCAAGGT | 836 | ATAGCCCACCCTC | 837 |
| (BTG2) | AA944156 | AAAAGG | | CAAAAACG | |
| CYP2B2 | M13234; | TGCTCAAGTACCCCCA | 838 | CAAATGCGCTTTC | 839 |
| | J00728 | TGTCA | | CTGTGGA | |
| AH- | AF082125; | TTCTTTCCACCCCAAT | 840 | CTGCATGCTTCTG | 841 |
| Receptor | AF082124 | TCCC | | ATGTCTTCG | |
| IGEBP-1 | M58634 | TTCTTTCCACCCCAAT | 842 | CTGCATGCTTCTG | 843 |
| | | TCCC | | ATGTCTTCG | |
| Amyloid_{—} | X07648 | ACACATGGCCAGAGTT | 844 | TCTTGAATCTCCT | 845 |
| A4 | | GAAGCC | | CAGCCACGG | |
| Gluta- | U73174 | CATGATCACGTGGATT | 846 | GAACCCATCACTG | 847 |
| thione | | ACGGC | | GTTATCCCC | |
| reductase | | | | | |
| Carboxyl | AB010635 | CAACATGCACCCAGCT | 848 | AGTCTTGGTCCAG | 849 |
| esterase | | ATTTCA | | AACTGCAGC | |
| CYP3A1 | D13912 | CTTTCCTTTGTCCTGC | 850 | TCAATGCTGCCCT | 851 |
| | | ATTCCC | | TGTTCTCC | |
| CYP9B | L00320 | CAACCCTTGATGACCG | 852 | CGCCAAGACAAAT | 853 |
| | | CAGTA | | GTGCTTTC | |
| UDP- | RNUD2A1 | GAGCCGTCTTCTGGAT | 854 | GGTCCCAACGCTG | 855 |
| glucuro- | 0, | CGAGTA | | TCTTCTTTT | |
| nosyl- | M35086; | | | | |
| transfer- | J05482 | | | | |
| ase 2B | | | | | |
| EGR1 | AF023087; | CAAAGCCAAGCAAACC | 856 | TCACGATTGCACA | 857 |
| (Krox24) | M18416; | AATGG | | TGTCCAGC | |
| | U7539; | | | | |
| | U75398; | | | | |
| | AI176662; | | | | |
| | RNNGFIA | | | | |
| GAPDH | P04797 | CCCAGAACATCATCCC | 858 | ATGTAGGCCATGA | 859 |
| | | TGCATC | | GGTCCACCA | |

TABLE 7
The accession numbers refer to GenBank.

| Affymetrix ID | Discrimination | Acc Number | SEQ ID NO |
|---|---|---|---|
| J03588_at | direct acting vs all other classes | J03588 | 681 |
| M13100cds#2_s_at | direct acting vs all other classes | M13100 | 682 |
| rc_AA800054_at | direct acting vs all other classes | AA800054 | 683 |
| rc_AI178750_at | direct acting vs all other classes | AI178750 | 684 |
| X53581cds#3_f_at | direct acting vs all other classes | X53581 | 685 |
| X58465mRNA_g_at | direct acting vs all other classes | X58465 | 686 |
| D78308_g_at | steatotic vs all other classes | D78308 | 687 |
| K00996mRNA_s_at | steatotic vs all other classes | K00996 | 688 |
| M94918mRNA_f_at | steatotic vs all other classes | M94918 | 689 |
| rc_AA892888_g_at | steatotic vs all other classes | AA892888 | 690 |
| rc_AA946503_at | steatotic vs all other classes | AA946503 | 691 |
| U88036_at | steatotic vs all other classes | U88036 | 692 |
| AF038870_at | cholestatic vs all other classes | AF038870 | 693 |
| AF076183_at | cholestatic vs all other classes | AF076183 | 694 |
| D00753_at | cholestatic vs all other classes | D00753 | 695 |
| J00738_s_at | cholestatic vs all other classes | J00738 | 696 |
| J03588_at | cholestatic vs all other classes | J03588 | 697 |
| K01932_f_at | cholestatic vs all other classes | K01932 | 698 |
| L27843_s_at | cholestatic vs all other classes | L27843 | 699 |
| M11670_at | cholestatic vs all other classes | M11670 | 700 |
| M15327_at | cholestatic vs all other classes | M15327 | 701 |
| rc_AA799899_i_at | cholestatic vs all other classes | AA799899 | 702 |
| rc_AA858673_at | cholestatic vs all other classes | AA858673 | 703 |
| rc_AA891220_at | cholestatic vs all other classes | AA891220 | 704 |
| rc_AA892333_at | cholestatic vs all other classes | AA892333 | 705 |
| rc_AA892775_at | cholestatic vs all other classes | AA892775 | 706 |
| rc_AA945143_at | cholestatic vs all other classes | AA945143 | 707 |
| rc_AA945321_at | cholestatic vs all other classes | AA945321 | 708 |
| rc_AI007820_s_at | cholestatic vs all other classes | AI007820 | 709 |
| rc_AI104524_s_at | cholestatic vs all other classes | AI104524 | 710 |
| rc_AI228674_s_at | cholestatic vs all other classes | AI228674 | 711 |
| rc_AI232087_at | cholestatic vs all other classes | AI232087 | 712 |
| X15734_at | cholestatic vs all other classes | X15734 | 713 |
| AB008424_s_at | non-toxic vs all other classes | AB008424 | 714 |
| AF045464_s_at | non-toxic vs all other classes | AF045464 | 715 |
| D78308_at | non-toxic vs all other classes | D78308 | 716 |
| J01435cds#8_s_at | non-toxic vs all other classes | J01435 | 717 |
| K01932_f_at | non-toxic vs all other classes | K01932 | 718 |
| M11794cds#2_f_at | non-toxic vs all other classes | M11794 | 719 |
| M13100cds#2_s_at | non-toxic vs all other classes | M13100 | 720 |
| M20131cds_s_at | non-toxic vs all other classes | M20131 | 721 |
| M64733mRNA_s_at | non-toxic vs all other classes | M64733 | 722 |
| rc_AA800054_at | non-toxic vs all other classes | AA800054 | 723 |
| rc_AA817964_s_at | non-toxic vs all other classes | AA817964 | 724 |
| rc_AA945054_s_at | non-toxic vs all other classes | AA945054 | 725 |
| rc_AA945169_at | non-toxic vs all other classes | AA945169 | 726 |
| rc_AI104679_s_at | non-toxic vs all other classes | AI104679 | 727 |
| rc_AI179012_s_at | non-toxic vs all other classes | AI179012 | 728 |
| rc_AI236795_s_at | non-toxic vs all other classes | AI236795 | 729 |
| S72505_f_at | non-toxic vs all other classes | S72505 | 730 |
| X03468_at | non-toxic vs all other classes | X03468 | 731 |
| X07467_at | non-toxic vs all other classes | X07467 | 732 |
| AB008807_g_at | controls vs all other classes | AB008807 | 733 |
| D00362_s_at | controls vs all other classes | D00362 | 734 |
| D00913_g_at | controls vs all other classes | D00913 | 735 |
| D25224_at | controls vs all other classes | D25224 | 736 |
| D25224_g_at | controls vs all other classes | D25224 | 737 |
| D43964_at | controls vs all other classes | D43964 | 738 |
| E01184cds_s_at | controls vs all other classes | E01184 | 739 |
| H32189_s_at | controls vs all other classes | H32189 | 740 |
| J00728cds_f_at | controls vs all other classes | J00728 | 741 |
| J02596cds_g_at | controls vs all other classes | J02596 | 742 |
| L37333_s_at | controls vs all other classes | L37333 | 743 |
| M11670_at | controls vs all other classes | M11670 | 744 |
| M15481_at | controls vs all other classes | M15481 | 745 |
| M20629_s_at | controls vs all other classes | M20629 | 746 |
| M28255_s_at | controls vs all other classes | M28255 | 747 |
| M31363mRNA_f_at | controls vs all other classes | M31363 | 748 |
| M58041_s_at | controls vs all other classes | M58041 | 749 |
| M64733mRNA_s_at | controls vs all other classes | M64733 | 750 |
| M76767_s_at | controls vs all other classes | M76767 | 751 |
| rc_AA800318_at | controls vs all other classes | AA800318 | 752 |
| rc_AA858673_at | controls vs all other classes | AA858673 | 753 |
| rc_AA860062_g_at | controls vs all other classes | AA860062 | 754 |
| rc_AA875107_at | controls vs all other classes | AA875107 | 755 |
| rc_AA891774_at | controls vs all other classes | AA891774 | 756 |
| rc_AA892775_at | controls vs all other classes | AA892775 | 757 |
| rc_AA892888_g_at | controls vs all other classes | AA892888 | 758 |
| rc_AA945143_at | controls vs all other classes | AA945143 | 759 |
| rc_AA946503_at | controls vs all other classes | AA946503 | 760 |
| rc_AI008641_at | controls vs all other classes | AI008641 | 761 |
| rc_AI011998_at | controls vs all other classes | AI011998 | 762 |
| rc_AI104524_s_at | controls vs all other classes | AI104524 | 763 |
| rc_AI136891_at | controls vs all other classes | AI136891 | 764 |
| rc_AI169372_g_at | controls vs all other classes | AI169372 | 765 |
| rc_AI172017_at | controls vs all other classes | AI172017 | 766 |
| rc_AI228674_s_at | controls vs all other classes | AI228674 | 767 |
| rc_AI232087_at | controls vs all other classes | AI232087 | 768 |
| S61868_g_at | controls vs all other classes | S61868 | 769 |
| S72505_f_at | controls vs all other classes | S72505 | 770 |
| S76779_s_at | controls vs all other classes | S76779 | 771 |
| X15096cds_s_at | controls vs all other classes | X15096 | 772 |
| X15512_at | controls vs all other classes | X15512 | 773 |
| X56325mRNA_s_at | controls vs all other classes | X56325 | 774 |
| X57432cds_s_at | controls vs all other classes | X57432 | 775 |
| X74549_at | controls vs all other classes | X74549 | 776 |
| X76456cds_at | controls vs all other classes | X76456 | 777 |
| X79081mRNA_f_at | controls vs all other classes | X79081 | 778 |

TABLE 8
The accession numbers refer to GenBank.

| | | | SEQ |
|---|---|---|---|
| Affymetrix ID | Discriminator | Acc Number | ID NO. |
| D25224_g_at | direct acting vs controls | D25224 | 779 |
| E01184cds_s_at | direct acting vs controls | E01184 | 780 |
| J02585_at | direct acting vs controls | J02585 | 781 |
| J02597cds_s_at | direct acting vs controls | J02597 | 782 |
| J03588_at | direct acting vs controls | J03588 | 783 |
| L19998_at | direct acting vs controls | L19998 | 784 |
| M13100cds#2_s_at | direct acting vs controls | M13100 | 785 |
| M94548_at | direct acting vs controls | M94548 | 786 |
| rc_AA800054_at | direct acting vs controls | AA800054 | 787 |
| rc_AI231807_g_at | direct acting vs controls | AI231807 | 788 |
| S76489_s_at | direct acting vs controls | S76489 | 789 |
| X53581cds#3_f_at | direct acting vs controls | X53581 | 790 |
| X57432cds_s_at | direct acting vs controls | X57432 | 791 |
| X58465mRNA_g_at | direct acting vs controls | X58465 | 792 |
| L00320cds_f_at | steatotic vs controls | L00320 | 793 |
| rc_AA946503_at | steatotic vs controls | AA946503 | 794 |
| X56325mRNA_s_at | steatotic vs controls | X56325 | 795 |
| AF038870_at | cholestatic vs controls | AF038870 | 796 |
| AF076183_at | cholestatic vs controls | AF076183 | 797 |
| D89375_s_at | cholestatic vs controls | D89375 | 798 |
| J00738_s_at | cholestatic vs controls | J00738 | 799 |
| J01435cds#1_s_at | cholestatic vs controls | J01435 | 800 |
| J03588_at | cholestatic vs controls | J03588 | 801 |
| J03863_at | cholestatic vs controls | J03863 | 802 |
| K01932_f_at | cholestatic vs controls | K01932 | 803 |
| K01934mRNA#2_at | cholestatic vs controls | K01934 | 804 |
| L27843_s_at | cholestatic vs controls | L27843 | 805 |
| M10068mRNA_s_at | cholestatic vs controls | M10068 | 806 |
| M11670_at | cholestatic vs controls | M11670 | 807 |
| M13100cds#3_f_at | cholestatic vs controls | M13100 | 808 |
| M14775_s_at | cholestatic vs controls | M14775 | 809 |
| M15327_at | cholestatic vs controls | M15327 | 810 |
| M20629_s_at | cholestatic vs controls | M20629 | 811 |
| M31018_f_at | cholestatic vs controls | M31018 | 812 |
| M34331_g_at | cholestatic vs controls | M34331 | 813 |
| M57718mRNA_s_at | cholestatic vs controls | M57718 | 814 |
| rc_AA800318_at | cholestatic vs controls | AA800318 | 815 |
| rc_AA858673_at | cholestatic vs controls | AA858673 | 816 |
| rc_AA859372_s_at | cholestatic vs controls | AA859372 | 817 |
| rc_AA945143_at | cholestatic vs controls | AA945143 | 818 |
| rc_AA945321_at | cholestatic vs controls | AA945321 | 819 |
| rc_AI072634_at | cholestatic vs controls | AI072634 | 820 |
| rc_AI102562_at | cholestatic vs controls | AI102562 | 821 |
| rc_AI104524_s_at | cholestatic vs controls | AI104524 | 822 |
| rc_AI105448_at | cholestatic vs controls | AI105448 | 823 |
| rc_AI228674_s_at | cholestatic vs controls | AI228674 | 824 |
| S76489_s_at | cholestatic vs controls | S76489 | 825 |
| X04979_at | cholestatic vs controls | X04979 | 826 |
| X15734_at | cholestatic vs controls | X15734 | 827 |
| X86561cds#2_at | cholestatic vs controls | X86561 | 828 |
| Y07704_at | cholestatic vs controls | Y07704 | 829 |

TABLE 9
Regualtion of GADD-family genes assessed by RT-PCR.

| Compound | Toxic manifestation | Dose amount | | Sampling time (Hours) | 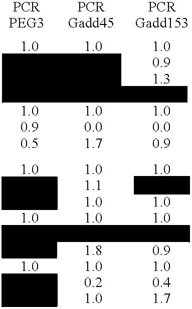 |
|---|---|---|---|---|---|
| Control (vehicle) Thioacetamide Thioacetamide Thioacetamide Control (vehicle) Thioacetamide Thioacetamide | Control Direct Acting Direct Acting Direct Acting Control Direct Acting Direct Acting | 0 2 10 50 0 2 10 | mg/kg mg/kg mg/kg mg/kg mg/kg mg/kg mg/kg | 6 6 6 6 24 24 24 | 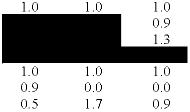 |
| Control (vehicle) Hydrazine Hydrazine Control (vehicle) Hydrazine Hydrazine Control (vehicle) Hydrazine Hydrazine | Control Direct Acting Direct Acting Control Direct Acting Direct Acting Control Direct Acting Direct | | | | |
| # Acting | 0 10 60 0 10 60 0 10 60 | mg/kg mg/kg mg/kg mg/kg mg/kg mg/kg mg/kg mg/kg mg/kg | 2 2 2 6 6 6 24 24 24 | 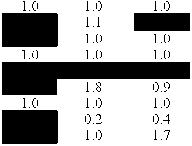 | |
| Control (vehicle) 1,2-dichlorobenzene 1,4-dichlorobenzene Control (vehicle) 1,2-dichlorobenzene 1,4-dichlorobenzene | Control Direct Acting Non-Toxic Control Direct Acting Non-Toxic | 0 4500 4500 0 4500 4500 | mmol/kg mmol/kg mmol/kg mmol/kg mmol/kg mmol/kg | 2 2 2 6 6 6 | 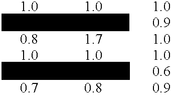 |
| Control (vehicle) 1,2-dichlorobenzene 1,4-dichlorobenzene | Control Direct Acting Non-Toxic | 0 4500 4500 | mmol/kg mmol/kg mmol/kg | 24 24 24 | 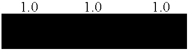 |

Shaded cells represent significant induction (threshold usually 2-fold induction).

TABLE 10
EGR-1 induction by Tasmar and Dinitrophenol.

| | | | Sampling | |
|---|---|---|---|---|
| | | Dose | time | |
| Compound | Toxic manifestation | Amount $ | (Hours)$ | PCR EGR-1 |
| Control (vehicle) | Control | 0 mg/kg | 3 | 1.0 |
| Tolcapone (Tasmar) | Human hepatotoxin | 300 mg/kg | 3 | 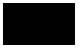 |
| Entacapone | Unknown | 600 mg/kg | 3 | 1.0 |
| Dinitrophenol | Mitochondrial uncoupler | 10 mg/kg | 3 | 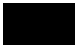 |
| Dinitrophenol | Mitochondrial uncoupler | 30 mg/kg | 3 | 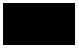 |

§: The compounds were administered to the experimental animals three times, every 12 Hours. Animals were sacrificed 3 hours after the last administration. Shaded cells represent significant induction (threshold usually 2-fold induction).

What is claimed is:

1. A method of predicting the mechanism of toxicity of a compound comprising detecting the level of expression in a tissue or cell sample exposed to the compound of one or more genes from Table 3, wherein differential expression of the one or more genes in Table 3 is associated with a specific mechanism of toxicity.

2. The method according to claim 1, wherein the expression levels of at least 2 genes from Table 3 are detected.

3. The method according to claim 1, wherein the expression levels of at least 5 genes from Table 3 are detected.

4. The method according to claim 1, wherein the expression levels of at least 10 genes from Table 3 are detected.

5. The method according to claim 1, wherein the expression levels of nearly all genes from Table 3 are detected.

6. The method according to claim 1, wherein the expression levels of all genes from Table 3 are detected.

7. The method according to claim 1, wherein the level of expression is detected by an amplification, hybridization or reporter gene assay.
